(19) United States
(12) Patent Application Publication
(10) Pub. No.: US 2022/0199265 A1
(43) Pub. Date: Jun. 23, 2022

(54) METHODS, SYSTEMS, APPARATUSES, AND DEVICES FOR MANAGING A SPREAD OF INFECTIOUS DISEASES AMONG USERS

(71) Applicant: Martin Tadandja, Wabern (CH)

(72) Inventor: Martin Tadandja, Wabern (CH)

(21) Appl. No.: 17/521,421

(22) Filed: Nov. 8, 2021

Related U.S. Application Data
(60) Provisional application No. 63/129,350, filed on Dec. 22, 2020.

(51) Int. Cl. G16H 50/80; H04W 4/029

(52) U.S. Cl. CPC G16H 50/80; H04W 4/029

(57) ABSTRACT

Disclosed herein is a device for managing a spread of infectious diseases among users. Further, the device comprises a communication device, a location sensor, a pathogen tracer microcontroller device, an output device, and a storage device. Further, the communication device receives device data from devices. Further, the location sensor generates location data based on a location of the device corresponding to a time period. Further, the pathogen tracer microcontroller device analyzes the device data and the location data, determines a spatiotemporal proximity of the device to the devices, compare the spatiotemporal proximity with a predetermined spatiotemporal proximity, determines a risk of the spread of a pathogen causing an infectious disease between the users, and generates an alert. Further, the output device produces alert signals based on the alert. Further, the storage device stores the alert and a status of a user corresponding to the infectious disease.

The current application claims a priority to the U.S. Provisional Patent application Ser. No. 63/129,350 filed on Dec. 22, 2020.

### FIELD OF THE INVENTION

Generally, the present disclosure relates to the field of data processing. More specifically, the present disclosure relates to methods, systems, apparatuses, and devices for managing a spread of infectious diseases among users.

### BACKGROUND OF THE INVENTION

The field of data processing is technologically important to several industries, business organizations, and/or individuals.

Over the last century, there has been an alarming increase in the number, frequency, and diversity of zoonotic disease outbreaks. Caused by the spillover of pathogens from animal hosts to people, these events may have more than tripled in the last decade, with the number of new zoonotic diseases infecting people quadrupling over the same period.

Today, 60% of emerging infectious diseases in humans are zoonotic. Animal pathogens may infect humans directly through contact with wild or domestic animals or indirectly by transmission through intermediate hosts. These intermediate hosts act as “mixing vessels” that may lead to the genetic variation of diseases, enabling them to infect humans.

And in modern times, contact with animals has dramatically increased, accelerating the risk of zoonotic disease outbreaks in humans.

Although new zoonotic diseases have posed a grave threat to human health around the world throughout the ages, in this modern traveling age they are ever more dangerous. Indeed, our way of life drives more frequent contact between humans and dangerous animal pathogens as well as contact with a wider variety of species, resulting in the emergence of new forms of diseases in humans.

When natural ecosystems like forests remain intact, interactions between human populations and wild host species are limited. As a result, viruses circulate with limited crossing over into humans. Similarly, wild host species have fewer interactions with domesticated animals and livestock, which generally live near humans. It is, therefore, less likely for domestic animals and livestock to become intermediate hosts of these diseases.

And while vaccine development is important, pathogens may leap from animals to humans much faster than scientists may develop vaccines and treatments. Controlling and eradicating zoonotic diseases in animals is more effective than in humans and it is far cheaper to invest in the prevention of infectious disease outbreaks than to deal with the consequences of a pandemic.

The only way out, according to one specialist is to adopt the One Health concept. Introduced at the beginning of the 2000s, it summarized the idea that human health and animal health are interdependent and bound to the health of the ecosystems in which they exist.

Consequently, global strategies to prevent and control pathogens must be developed. These should be coordinated at the human-animal-ecosystems interface and applied at the national, regional, and global levels, through the implementation of appropriate policies.

But even then, it is not stopping the cause, just monitoring the inevitable. The current Coronavirus pandemic is an example that just can't be ignored. The spillovers may continue to happen as long as humankind keeps threatening the health of our ecosystems. The health of our planet and animals has become a reflection of public health and needs to be addressed urgently. (Rozenbaum, 2020)

Existing techniques for managing a spread of infectious diseases among users are deficient with regard to several aspects. Further, infectious diseases are threatening the human culture more so than in the past. The current approach of hoping for vaccines and treatments is too slow, carries uncertainties, and causes a lot of damage to the economy. Further, a disease must find healthy living creatures to contaminate. Furthermore, current technologies do not prevent the disease to find other healthy living creatures to contaminate (or spread).

Therefore, there is a need for improved methods, systems, apparatuses, and devices for managing a spread of infectious diseases among users that may overcome one or more of the above-mentioned problems and/or limitations.

### SUMMARY OF THE INVENTION

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

Disclosed herein is a device for managing a spread of infectious diseases among users, in accordance with some embodiments. Further, the device may be associated with a user. Further, the device may include a communication device, a location sensor, a pathogen tracer microcontroller device, an output device, and storage device. Further, the communication device may be configured for receiving one or more device data from one or more devices associated with one or more users. Further, the one or more device data may include one or more location data associated with one or more locations of the one or more devices corresponding to one or more time periods. Further, the location sensor may be configured for generating location data based on a location of the device corresponding to a time period. Further, the pathogen tracer microcontroller device may be communicatively coupled with the communication device. Further, the pathogen tracer microcontroller device may be configured for analyzing the location data and the one or more location data. Further, the pathogen tracer microcontroller device may be configured for determining a spatiotemporal proximity of the device to the one or more devices based on the analyzing. Further, the pathogen tracer microcontroller device may be configured for comparing the spatiotemporal proximity with a predetermined spatiotemporal proximity based on the determining of the spatiotemporal proximity. Further, the pathogen tracer microcontroller device may be configured for determining a risk of the spread of a pathogen causing an infectious disease between the user and the one or more users based on the comparing. Further, the pathogen tracer microcontroller device may be configured for generating an alert based on the determining of the risk. Further, the output device may be communicatively coupled with the pathogen tracer microcontroller device. Further, the output device may be configured for producing one or more alert signals based on the alert. Further, the storage device may be communicatively coupled with the pathogen tracer microcontroller device. Further, the storage device may be configured for storing the alert. Further, the storage device may be configured for storing a status of the user corresponding to the infectious disease.

Further disclosed herein is a device for managing a spread of infectious diseases among users, in accordance with some embodiments. Further, the device may be associated with a user. Further, the device may include a communication device, a location sensor, a pathogen tracer microcontroller device, an output device, and a storage device. Further, the communication device may be configured for receiving one or more device data from one or more devices associated with one or more users. Further, the one or more device data may include one or more location data associated with one or more locations of the one or more devices corresponding to one or more time periods. Further, the location sensor may be configured for generating location data based on a location of the device corresponding to a time period. Further, the pathogen tracer microcontroller device may be communicatively coupled with the communication device. Further, the pathogen tracer microcontroller device may be configured for analyzing the location data and the one or more location data. Further, the pathogen tracer microcontroller device may be configured for determining a spatiotemporal proximity of the device to the one or more devices based on the analyzing. Further, the pathogen tracer microcontroller device may be configured for comparing the spatiotemporal proximity with a predetermined spatiotemporal proximity based on the determining of the spatiotemporal proximity. Further, the pathogen tracer microcontroller device may be configured for determining a risk of the spread of a pathogen causing an infectious disease between the user and the one or more users based on the comparing. Further, the pathogen tracer microcontroller device may be configured for identifying one or more first users from the one or more users based on the comparing. Further, the infectious disease may be likely to spread between the user and the one or more first users. Further, the pathogen tracer microcontroller device may be configured for generating an alert based on the determining of the risk and the identifying. Further, the output device may be communicatively coupled with the pathogen tracer microcontroller device. Further, the output device may be configured for producing one or more alert signals based on the alert. Further, the storage device may be communicatively coupled with the pathogen tracer microcontroller device. Further, the storage device may be configured for storing the alert. Further, the storage device may be configured for storing a status of the user corresponding to the infectious disease.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTION OF THE DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 is a block diagram of a device for managing a spread of infectious diseases among users, in accordance with some embodiments.

FIG. 3 is a block diagram of a device for managing a spread of infectious diseases among users, in accordance with some embodiments.

FIG. 4 is a block diagram of a system for managing a spread of infectious diseases among users, in accordance with some embodiments.

FIG. 5 is a flow chart of a method for managing a spread of infectious diseases among users, in accordance with some embodiments.

FIG. 6 is a block diagram of a system for managing a spread of infectious diseases among users, in accordance with some embodiments.

FIG. 7 is a schematic of a system for managing a spread of infectious diseases among users, in accordance with some embodiments.

FIG. 8 illustrates a plurality of Trat devices associated with the disclosed system, in accordance with some embodiments.

FIG. 9 is a graphical representation illustrating a time window and number of receiver slots for a TraT device associated with the disclosed system, in accordance with some embodiments.

FIG. 10 is a schematic of a system for managing a spread of infectious diseases among users, in accordance with some embodiments.

FIG. 11 is a block diagram of a computing device for implementing the methods disclosed herein, in accordance with some embodiments.

### DETAIL DESCRIPTIONS OF THE INVENTION

As a preliminary matter, it may readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, may be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of methods, system, devices, and apparatuses for managing a spread of infectious diseases among users, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor, and at least one actuator. Examples of the one or more client devices and/or the server computer may include, a desktop computer, a laptop computer, a tablet computer, a personal digital assistant, a portable electronic device, a wearable computer, a smartphone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server, etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface, etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third-party database, a public database, a private database, and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role-based access control, and so on.

Further, one or more steps of the method disclosed herein may be initiated, maintained, controlled, and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal, or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer, etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera, and so on. Likewise, the one or more steps may include receiving, using the communication device, the one or more embodied characteristics from one or more biometric sensors.

Further, one or more steps of the method may be automatically initiated, maintained, and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device, etc.) corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor, etc.), and a biometric sensor (e.g. a fingerprint sensor)

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data, and any intermediate data therebetween corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

Overview:

The present disclosure describes methods, systems, devices, and apparatuses for managing a spread of infectious diseases among users. Further, citizens of democratic countries do not like to be monitored but to combat pandemic monitoring is essential. The disclosed device allows governments and private organization to monitor their population in case of infectious disease without the population fearing to live in a totalitarian state. Further, the device may be carried by each individual collects all encounters of the person. Once a person is contaminated with the disease, all the people he or she had contacts are informed. Further, no personal data may be involved. Further, no personal data may be stored in the device. Furthermore, people (or users) may be informed by the device if they are around contaminated people so that people may avoid them. The device may be designed to be hack-proof There is no human interaction with operating the device. Therefore, the disclosed device puts people on quarantine as faster as the virus spreads or faster. Further, components of the disclosed device may secure the privacy of the person on the process, device, and network level. Further, the disclosed device may be associated with a software platform that may be used to manage infectious diseases.

Further, infectious diseases are threatening the human culture more so than in the past. The current approach of hoping for vaccines and treatments is too slow, carries uncertainties, and causes a lot of damage to the economy. Further, a disease must find healthy living creatures to contaminate. Furthermore, current technologies do not prevent the disease to find other healthy living creatures to contaminate (or spread).

The disclosed device may be designed to propagate the isolation or quarantine of the population faster than the speed of spreading of the disease. The success of the disclosed device may be based solely on the length of time necessary to be in contact with an infected living creature and the length of time in which the infected living creature may infect other living creatures. The more these values are based on evidence-based medicine the higher is the success rate of this tool to exterminate or eliminate the infectious disease.

Another critical success factor is the estimate of unrecorded cases. The epidemiologist has to estimate the sample size such that the number of positive results of the random tests is large than the estimated number of unrecorded cases.

Governments and private organization may use the disclosed tool to make sure that the section of the population that does not want to be contaminated have a chance to achieve this goal. Furthermore, this section of the people using the disclosed device may have a gathering without abiding by the laws governing the disease.

Further, the disclosed device may not need personal or geographical data. Hence, the privacy of the participant is protected. The process of mapping a probe of a person to the identifier of the system should take place anonymously with the virus probe register device.

The proximity of a person to another person may be determined by the range of the electromagnetic waves. In this application, the range may be set by the medical professional.

Further, a program code of the Transmission Chain Breaker may reside in a programmable read-only memory. This means the off the self-computers are not used and a proprietary operating system is to be developed for all hardware devices.

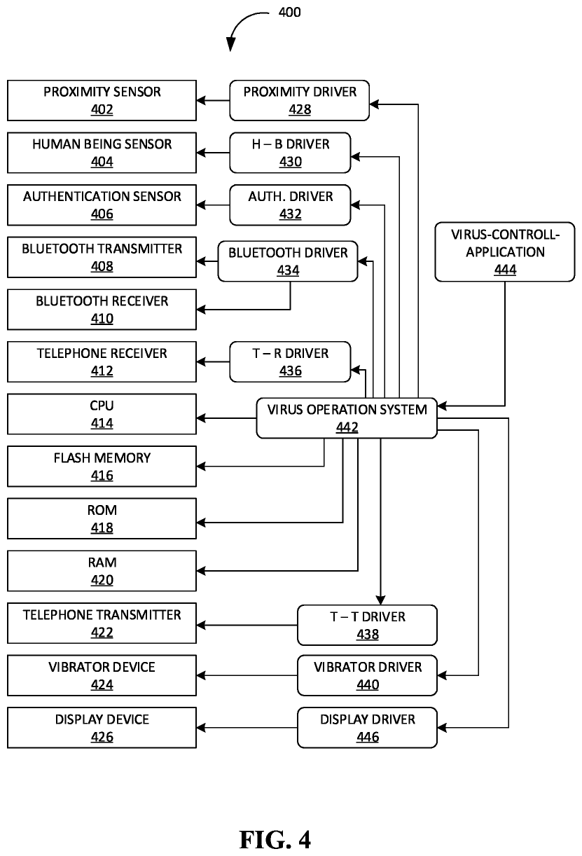

Further, as shown in FIG. 4, components of the system architecture may be mandatory except for the authentication sensor. Further, an authentication sensor is for the private sector.

Further, in an instance, organizers of mass events may stage mass events and assure the authorities that diseases law are not violated. Further, the organizer declares that wearing the device and being quarantine is a prerequisite to taking part in the event. Therefore, a buyer of the even ticker is sent for each ticket a device. All spectators agree to voluntary be in quarantine according to the incubation period. The length of the voluntary quarantine is defined by the incubation period. All spectators may only enter if the status of the device allows it. The spectators of the event keep the devices for another length of the incubation period before sending them back for a refund. The spectator is responsible to inform the organizer and send the device back should he or she test positive after the event. The return device allows the organizer to put those who encountered this person in quarantine. The data concerning the authentication may be only stored locally and cannot be accessed by anyone.

The specification for the virus operation system is defined by the command describing the algorithm.

Care has been taken so that the privacy of the people cannot be violated now and in the future. The disclosed device is designed to be hack-proof so that it is theoretically impossible to hack it no matter it is using the public telephone system. Even if it is hacked what are you going to do with telephone numbers with no personal data? A telephone directory has more information than this design system. The main goal of the disclosed device may be as efficient and effective at fighting the disease as the system of a totalitarian state but being in line with the western culture. There is only one event in which the system punishes the person. If the person does not turn up for a test, then he or she is put to quarantine. All his or her encounters are not punished. Instead of a beeper, a vibrator may be used with more discretion. The user must disable the mobile phone when the Transmission Tracer communicates with the Transmission Chain Breaker.

The programs counter be in the range of the Data Section and the programs are stored in PROM. No software public libraries may be used except the divers. In the worst case, the drivers have to be programmed from scratch. The programming may be monitored by civil rights organizations For the sake of discretion, the device of the person violating the law should vibrate and the other may also vibrate. This may enable the person violating the law to escape without being recognized. This carries the risk that a crowd of people could panic. On the other hand, the disclosed system may be designed to react differently in such circumstances to avoid a panic. This is an example of how the disclosed system is different from a system of a totalitarian state or informers' state.

The disclosed system may combat infectious diseases. The distribution of the disclosed devices may be done with no registration. This means the devices may be put on shelves at supermarkets, restaurants, movie houses, and so on. Anyone may pick it up and wear it. Furthermore, the disclosed system put the people in quarantine and isolation at least the speed at which the diseases spread. The disclosed system allows virtual isolation and virtual quarantine. An employee who may be in the quarantine may show up to work without the risk of contaminating the other employee as long as the social distancing is enforced. The disclosed system may enforce social distancing. These are the innovations. Last but not least the disclosed system differs from a system of a totalitarian state in that the system of a totalitarian state does not have the discretion functionality and performance.

Extermination or elimination of an infectious disease may only be achieved if the tools are applied nationwide. Anyone entering the country to stay long has to have been in quarantine for a given number of days. During that period the person has to wear the device for 16 hours or so per day. This may be monitored by a sensor that senses how long the device is worn. It is up to the government to define how fool-proof the system has to be. Carriers have to provide a section in their vehicles for those wearing the device. Tourists may have to leave the device at the border upon exiting the country. The deposited devices may be reused after a defined period.

Border crosses are the greatest challenge, but they have to wear this device too. Further, the sample size for people to be tested may contain the spreading of the disease. Agreements may be reached with border crosses to only have contacts with very close relatives. These relatives may also be given such a device.

Governments and private organizations may achieve one of the following goals. The first goal is a discriminated society where there is an infrastructure for both populations. This option allows the citizen the freedom of choice and could lead to 90% of the population embracing the system. The benefit of the person wearing the device is that person may interact like before the pandemic. The private section may embrace this system because it enables them to deliver the service their clients want. It may be difficult for a majority of the population to accept to live in difficult conditions than those embracing this system. Only the diehard may avoid using the disclosed system in the long run. The second goal is to exterminate the disease. This does not allow freedom of choice but is economical.

After a pandemic government and private organizations have to decide if the system should stay operational. This may allow it to combat the annual flu. Doctors may send early suspects of the flu for testing.

The population has to vote on the matter. If more than 60% of the population agrees on the matter, then mandatory usage is permitted. This policy is necessary to determine if persons who do not turn up for the test are punished or not. This voting allows us to increase the sample size according to because we may assume that no voters are not going to turn up. Furthermore, legislation has to be passed insuring that this system is only used for extermination or elimination of diseases. Violating this law must carry severe punishments and whistleblowers are guaranteed immunity and received a high amount of payment.

To be able to answer this question let's take a look at the waterfall methodology which divides the development process into requirements, analysis, design, implementation, testing, operation/deployment, and maintenance. After analyzing the different approaches to handling the COVID-19 Crises, all approaches had one fundamental error. Further, the approaches are all based on the assumption that a vaccine is necessary to overcome crises caused by infectious diseases. Therefore, tools may be needed that help us manage the crises until the vaccine is available. What is required is something that combats future infectious diseases at once and does not have to be developed once the disease is known. In other words, it's asking a pharmacologist to develop a vaccine for all unknown infectious diseases. He or she may probably consider this as an impossible task. If the people of a country may be convinced that vaccines are not the solution, but the disclosed system may be the future then vaccines are no more need. The disclosed system may have the potential to make vaccines obsolete.

What is new is the problem-solving approach and that is to substitute vaccines with another methodology that may instantaneously be applied once the new infectious disease is diagnosed as such. Secondly, the methodology may only inhibit the lives of the people who could have potentially caught the disease. Thirdly, the methodology has to allow the business to do business as usual. This means that governments do not have to order businesses to close because of the infected population.

Further, the disclosed device may include combat tools for extermination or elimination of the infectious disease. Further, the disclosed device may collect all encounters of the population using the virus Alarm Device. Further, the disclosed device may determine the infected population using the virus Alarm Device and virus Probe Register device. Further, the disclosed device may separate the infected population using the virus alarm device and the virus attacker. Further, the disclosed device may prevent the two populations from mixing using the virus Alarm Device and Virus Monitor.

The term Transmission tracer is a synonym of virus alarm device.

The term Transmission chain breaker is a synonym of virus attacker

The term Quarantine Monitor is a synonym of Virus Monitor

### Virus Control Application:

device_operation_mode is normal
advance_length_of_vibration is 120 seconds
WHILE battery Level suffice to secure system operation DO
/* Is a virus alarm Devic close near than social distance*/
Set Value of human_being_with_a n_virus-alarm-Device as False
IF proximity sensor notices another Virus-Alarm-Device THEN encountered-device-system
Set human_being_with_an_virus-alarm-Device as True
END IF
/* Informs an entry device about the health status */
IF proximity Senor notice an Entry Device THEN
Send virus-alarm device Status to Entry Device
IF virus-alarm-device Status is not free movement THEN
Turn vibrator on
END IF

### END IF

* Informs virus-alarm-device that person has attend Test*
IF proximity Senor notice a Virus Probe Register device THEN
Set value of the variable expired_show_up_time to current system date+200 years
END IF
* Use the human detection sensor to detect a person *
IF human_being_with_an_virus-alarm-Device is False THEN encountered-person-notification-system
END IF *
* Virus attacker informs virus alarm device to set Status to quarantine *
IF telephone receiver has message “Set virus-alarm-device Status to quarantine” law-enforcement-system
END IF
* Latency for determining the chain of infection *
IF (Telephone receiver has message chain_of_infection) THEN chain_of_infection=value_of in coming chain_of_infection
END IF
The user happens to be in the sample group for testing *
IF telephone receiver has message “Please go to a test center for a virus test” THEN Display the message “Please go to a test center for a virus test in x days or hours” in the display
Set value of the variable expired_show_up_time to current system date+x day or hours
END IF
* Put the person in to quarantine because did not show up at test center *
IF expired_show_up_time less than current system time THEN
Set virus-alarm-device Status to quarantine
Set value_of_begin_of_quarantine equal system time plus X hours
Set value_of end_of_quartantine equal system time+defined time for quarantine+X hours
END IF
* Handles all tampering activities of criminals *
* This is a customer specific requirement*
* Self-imposed quarantine *
GET quarantine_button value
IF quarantine_button is TRUE THEN
Set monitoring_wear_virus_alarm_device value is True
Get from authentication sensor current barometric-data
last barometric-data=current barometric
quarantine button value is false
END IF
* Monitoring the user for 14 day of self-imposed quarantine *
IF monitoring_wear_virus_alarm_device is True THEN
Get from authentication sensor current barometric-data
IF last barometric-data NOT EQUAL current barometric data THEN
Set virus-alarm-device Status to quarantine

Send to display the following message “Within X hours you have be in Quarantine because you temper with the device*
Set value_of_begin_of_quarantine equal system time plus X hours
Set value_of end of quarantine equal system time+incubation_period+X hours
ELSE
last barometric-data=current barometric
END IF
END IF
* End of quarantine or isolation *
IT system time greater than end_of_quarantine or than end_of_isolation THEN
Set monitoring_wear_virus_alarm_device is False
Set virus-alarm-device Status to free movement
END IF
* Manages all information to Display device *
IF Message on Display exceeds a defined time THEN
Delete Message
END IF
* Turns the vibrator of a person in isolation or quarantine x seconds in advance *
IF virus-alarm device status equal isolation OR virus-alarm device status equal quarantine THEN
advance_length_of_vibration equal a to be defined number of seconds
END IF
* Turns the vibrator off*
IF vibrator is vibrating longer than advance_length_of_vibration plus a defined time THEN
Turn vibrator off
advance_length_of_vibration equal 0 seconds.
END IF
* Send Virus-alarm of foreigner to Attacker”
IF inform_attacker_of_foreigner_device less than System time THEN
inform_attacker_of_foreigner_device=System time+24 hours
While telephone number on the flash Disk has not been read one DO
Read telephone_number on the flash Disk
IF telephone number is within range of Foreigner THEN
Send foreigner's telephone number and the telephone number of current alarm device
END DO
END IF
END WHILE

### Begin Encountered Device System:

SEND virus-alarm-device telephone number and virus-alarm-device Status
GET virus-alarm-device telephone number of encountered virus-alarm-device and virus-alarm-device Status of encountered virus-alarm-device
* Checks if the encountered person may transmit the virus *
IF virus-alarm-device Status of encountered virus-alarm-device equal Isolation OR equal Quarantine THEN

* Ensure that violated person may escape unnoticed*
End of Wait time=advance_length_of_vibration+system time telephone number to wait=virus-alarm-device telephone number of encountered virus-alarm-device escaping telephone number=virus-alarm-device telephone number of encountered virus-alarm-device
WHILE system time is less than End of wait time DO
GET virus-alarm-device telephone number of encountered virus-alarm-device and virus-alarm-device Status of encountered virus-alarm-device
*The device driver hast to flash the telephone after it is read *
IF virus-alarm-device telephone number of encountered virus-alarm-device equal escaping telephone number AND system time is greater than End of wait time THEN
Send message to Display “Safety distance is violated”
Turn vibrator on
END IF
END WHILE
END IF
* Alarms the user of the device *
IF virus-alarm device status is quarantine OR isolation THEN
Turn vibrator on
END IF
* Was the encounter long enough to transmit the virus *
IF Virus-Alarm Device Status of encountered virus-alarm-device equal free movement THEN Infection-Management
END IF
END Encountered-Device-System

### Infection Management:

IF telephone number of encountered virus-alarm-device is not in Flash Memory of specific day THEN
SAVE telephone number of encountered virus-alarm-device in RAM of the specific day with TIME STAMP
SORT ASCENDING according to telephone number and time stamp of the specific day

IF telephone number of encountered virus-alarm-device in the RAM of the specific day AND the following telephone number of encountered virus-alarm-device in RAM is equal THEN
SUBTRACT Time Stamp of first telephone number of encountered-virus-alarm device from the second telephone number of the encountered virus-alarm device in RAM Stack of the specific day IF result of subtraction is greater time necessary for infection THEN
SAVE telephone number of encountered virus-alarm-device in Flash Memory of the specific day.
DELETE all the telephone number of encountered virus-alarm-device in the Ram of the specific day
ELSE
DELETE the second telephone number of the encountered virus-alarm device from the RAM Stack of the specific day
END IF
END IF

### Law Enforcement System:

IF telephone call is from virus-attacker THEN
* Preparing for Isolation of USER *
Send to display the following message “With in X hours you have be in isolation” Set value_of_begin_of_isolation equal system time plus X hours
Set value_of end_of_isolation equal system time+incubation_period+X hours chain_of_infection=0
* Put all person encountered during time of incubation into quarantine *
While telephone number on flash disk as not be read once DO
Send message to alarm device telephone number on flash disk to set status of that alarm-device to quarantine
Send value_of chain_of_infection to alarm device of telephone number on flash disk
END WHILE
END IF
* Put all person encouterd during time of incubation in quarantine after latency time *
IF telephone call is from virus-alarm-device THEN
Send to display the following message “With in X hours you have be in Quarantine*
Set value of begin_of_quarantine equal system time plus X hours
Set value of end_of_quartantine equal system time+incubation_period+X hours
* Informs all alarm device which need to have the status quarantine *
Get chain_of_infection
chain_of_infection=chain_of_infection+1
Begin_date_of_latency=Search in the list of telephone on the flash disk the oldest date).
End_date_of_latency=Begin_date_of_latency+Chain of infection times latency.
While telephone number on flash disk as not be read once DO
Read telephone number on flash disk with Time Stamp
IF Time Stamp of telephone number on flash disk is greater than End_date_of_latency THEN
Send message to alarm device telephone number on flash disk to set Status of that alarm-device to quarantine
Send value of chain_of_infection to alarm device of telephone number on flash disk
END IF
END WHILE
END IF

### Encountered Person Notification System:

DO
Turn human being sensor on
IF human being sensor notice a human being THEN
Send the message “human being is not carrying a virus-alarm-device” to display
END IF
Turn human being senor off
END DO

### Virus Attacker Application:

While NOT shut down
* Communication positive Test to virus-alarm device*
IF a telephone call is received from Testing Unit of a hospital THEN
Send to the telephone number received from the Testing Unit the message “Set values of Virus-alarm Device Status to Isolation “search with the telephone number received from Testing Unit of hospital for a telephone number in the lists foreigner
IF search is successful THEN
Send to the telephone number found the message “Set values of Virus-alarm Device Status of the deceive given to a foreigner to Isolation *The trick is to send all citizen who encounter this foreigner in quarantine *
END IF
* Back tracing foreigner who might have infected a citizens *
IF a telephone call is received from a virus-alarm device with the list of telephone number of alarm devices given to foreigner who are no more in the country * THEN
Store these lists foreigner
Sort these list foreigner according to the telephone number of the citizens who meet this foreigner
END IF
* Generate a sample of telephone number to send people for test*
IF population testing value is True THEN
Generate randomly a sample of n Telephone numbers where value of virus_infection variable is false and value of the variable been to test is false set population_testing value to False
Send to all Telephone numbers in the sample the following the message “Please go to a test center for a virus test”
Set the value of been to test to True for all these Telephone numbers
END IF
End While

### Virus Monitoring Application:

WHILE NOT Shut shutdown
IF received virus-alarm-device Status is free movement THEN allow human being to enter
END IF
End While

The disclosed device may be placed at homes, offices, and public locations. It prohibits the communication to the attacker server or other virus alarm servers over the public telephone network.

Over the last century, there has been an alarming increase in the number, frequency, and diversity of zoonotic disease outbreaks. Caused by the spillover of pathogens from animal hosts to people, these events may have more than tripled in the last decade, with the number of new zoonotic diseases infecting people quadrupling over the same period.

Today, 60% of emerging infectious diseases in humans are zoonotic. Animal pathogens may infect humans directly through contact with wild or domestic animals or indirectly by transmission through intermediate hosts. These intermediate hosts act as “mixing vessels” that may lead to the genetic variation of diseases, enabling them to infect humans.

And in modern times, contact with animals has dramatically increased, accelerating the risk of zoonotic disease outbreaks in humans.

Although new zoonotic diseases have posed a grave threat to human health around the world throughout the ages, in this modern traveling age they are ever more dangerous. Indeed, our way of life drives more frequent contact between humans and dangerous animal pathogens as well as contact with a wider variety of species, resulting in the emergence of new forms of diseases in humans.

When natural ecosystems like forests remain intact, interactions between human populations and wild host species are limited. As a result, viruses circulate with limited crossing over into humans. Similarly, wild host species have fewer interactions with domesticated animals and livestock, which generally live near humans. It is, therefore, less likely for domestic animals and livestock to become intermediate hosts of these diseases.

But once the forests start to disappear, populations come in closer contact, and disease spreads. Optimal rates of spillover occur once 40 percent of the forest cover disappears.

Greater biodiversity of species in a natural ecosystem like a forest may hinder disease transmission. This may be attributable to what scientists call the “dilution effect,” which makes it more difficult for a single pathogen to spread rapidly or to dominate.

So, if populations are separate, virus pools remain distinct. But unfortunately, wildlife trade, unsustainable food systems, and deforestation have brought animals and humans in very close proximity over the last decades.

Bats, that used to live far from humans in a cave, now roam cities.

The latest COVID-19 pandemic is only the most recent example of how dangerous animal viruses may be on a global scale. Let's not forget how the Ebola virus went from bats to monkeys to humans in the 1970s causing an incredibly deadly hemorrhagic fever and still causes major outbreaks today.

Historically rabies, which causes an inflammation of the brain, went from most dogs to humans around 2000 BC but is still killing thousands every year. And let's not omit AIDS, which creates an immune deficiency and went from monkeys to humans in the early 20th century, and which has infected approximately 37.9 million people across the globe.

These are only a handful of examples showing how dangerous zoonotic diseases may be, and there are hundreds more out there. There are over 150 zoonotic diseases worldwide, which are transmitted to humans by both wild and domestic animal populations, 13 of which are responsible for 2.2 million deaths per year.

And while vaccine development is important, pathogens may leap from animals to humans much faster than scientists may develop vaccines and treatments. Controlling and eradicating zoonotic diseases in animals is more effective than in humans and it is far cheaper to invest in the prevention of infectious disease outbreaks than to deal with the consequences of a pandemic.

The only way out, according to one specialist is to adopt the One Health concept. Introduced at the beginning of the 2000s, it summarized the idea that human health and animal health are interdependent and bound to the health of the ecosystems in which they exist.

Consequently, global strategies to prevent and control pathogens must be developed. These should be coordinated at the human-animal-ecosystems interface and applied at the national, regional, and global levels, through the implementation of appropriate policies.

But even then, it is not stopping the cause, just monitoring the inevitable. The current Coronavirus pandemic is an example that just can't be ignored. The spillovers may continue to happen as long as humankind keeps threatening the health of our ecosystems. The health of our planet and animals has become a reflection of public health and needs to be addressed urgently. (Rozenbaum, 2020)

The concept used solves the technical, organizational, judicial, acceptance, and financial difficulty. No personal data, central data storage, and no staff for tracing are needed. Excellent service to help the person in quarantine and the populations may get rid of the plague are required. There are no free lunches. If 85 to 95% of the population is willing to put up a fight against zoonotic disease, then nothing stands in the way of successful implementation. Compared to today's solution of the Covid-19 pandemic, the state becomes predictable. The government does not thwart the plans of citizens with constant new measures. Citizens know how to plan to achieve maximum freedom. The COVID-19 pandemic has led to a dramatic loss of human life worldwide. It presents an unprecedented challenge to public health, food systems, and the world of work. This study aims to determine the technology and organization require to prevent zoonotic pandemics. These devices manage the elimination of zoonotic diseases.

The statement of the American experts on American circumstances dominates this document. Nevertheless, the solution is adaptable to the cultural requirement of other nations. Therefore, applicable in other countries

Furthermore, from General G Patton and Gina Raimondo's (secretary of commerce) statements, the developer concludes that willingness to fight is part of the culture of the US. The willingness to fight in a nation is indispensable for an elimination campaign. Therefore, an elimination campaign has the best chance of success in the US. Nevertheless, the developer advises other countries to try it with appropriate adaptation to their cultural conditions.

The audience is all public health persons, politicians, and military persons. In particular, the Secretaries of Health and Human Service and Defense should review the disclosed system with his staff.

An oppressive state may implement the disclosed system (or a health system) within months. A military slaughter of a country due to a zoonotic pandemic does not affect them. The oppressive state may, in this situation, strengthen its influence and access to other countries and strike, despite inferior weaponry.

No Democratic state with an army may afford not to implement this health response system. Anyway, the democratic state cannot implement it by pressure. The Achilles heel of the disclosed system is everyone must be ready to show the symptoms of an unknown disease to a doctor as soon as possible. It is not a contradiction that an oppressive state may impose it with pressure, and a democratic state cannot. The question is whether a state is willing to torture its citizens or not. Such a question does not apply to a democratic state. Dictators torture their people to achieve their goals.

My conviction is solving a problem is based on solving the economic problem. Based on this world view, the zoonotic pandemic is primarily an economic problem and secondarily perhaps a medical, social, etc. So, what economic problems are there.

Further, the zoonotic pandemic may cause all of these problems. The developer may not be satisfied with solving only one of these problems.

The Covid-19 pandemic and policy responses have made life much more uncertain for everyone—individuals, organizations, and governments. What impact does all this uncertainty have on the economy and how may policy-makers respond?

The Covid-19 pandemic and policy responses have led to a huge increase in economic uncertainty about the future. For how long may social distancing be practiced? What impact may social distancing have on our normal daily activities? may a vaccine be discovered?

Such questions create uncertainty for everyone:

For individuals, there is uncertainty about their future work prospects, their incomes, the value of their savings, and what they may be able to spend their money on.

For firms, there is uncertainty about who may buy their products, whether they may source essential inputs, and what changes they may have to make to their workplaces to accommodate social distancing requirements.

For governments, there is a large increase in uncertainty about future revenue and spending requirements, as well as how people and firms may behave and what effects that may have on policy.

What impact does all this uncertainty have on the economy? There is a large body of research evidence showing that increasing uncertainty has negative effects on the economy. This suggests that the economic effects of Covid-19 could be bigger and more persistent than the immediate disruption. This research also indicates that anything that governments may do to reduce uncertainty—by making clear policy statements and by providing insurance—might help to mitigate these effects (McMahon, 2020).

The economic problem is how to reduce the uncertainty to the level before the zoonotic pandemic. The impact of this increased insecurity due to zoonosis is devastating and must remain unique.

Any proposal to resolve a zoonotic pandemic on the scale of Covid-19 must lead to a reduction in uncertainty. Any solution to a zoonotic pandemic must only contain risks that the population faced before the pandemic.

Cross-referencing of quarterly economic and health data confirms the superiority of the elimination strategy in terms of anticipation. People in those countries benefit from a level of visibility enabling them to project their societies and economies into the future. In contrast, the course taken by the G10 countries has produced fluctuations, with the epidemic rebounding in the fourth quarter of 2020 everywhere except Japan, which is moving closer to Zero Covid. The mitigation strategy is causing them to seesaw, making it difficult to project into the future and thereby penalizing societies and economies. This is especially problematic for businesses that depend on significant social interaction, which have been closed for months, as representatives of the hotel, restaurant, culture, and recreation sectors have stated repeatedly. The Zero Covid strategy protects people and economies more effectively. In February 2021, Google searches for the word “Restaurant” were 64% lower than in February 2019. This decline was five times greater than in the Zero Covid countries, where restaurants are open and searches are down only 13% from February 2019. Participation in economic and social life is a function of people's confidence in being able to take part without running the risk of falling ill, contaminating others, or seeing health services overwhelmed. (Marques, 2021)

There are several required aspects of a disease that need to be fulfilled for a disease to be considered eradicable:
It needs to be and an infectious disease
We need to have ways to either prevent or treat the infection

For a disease to be eradicable it needs to be a disease you may “catch” from other humans or animals, that is it has to be infectious. Non-infectious diseases, such as heart disease or cancer, cannot be eradicated.

Without an effective treatment against a disease, there is no possibility of eradicating it. The treatment may be either preventative, such as vaccination, or curative, such as drugs that may eliminate the pathogen that causes the disease from its host. (Max Roser, 2018)

In addition, the key requirements there are many other aspects of the disease that should be considered in the efforts to eradicate it:
How many pathogens cause the disease?
Does the disease-causing pathogen have one or more hosts?
Are there any identifiable symptoms of the disease?
Has regional disease elimination proven possible?
Is the perceived disease burden high and is financial and political support available? (Max Roser, 2018)

Modern public health lumps all health threats together and seeks to deal with them through a general integrated system of services. Thus, programs such as injury prevention, hypertension management, and prenatal care are viewed the same way as infectious disease control. Yet, history and science tell us that infectious disease control is fundamentally different from other public health concerns (Richards & Rathbun, 1999)

French President Emmanuel Macron has ordered people to stay at home and is closing the country's land borders from 1200 (1100 GMT) on Tuesday. He said the country was at war with an invisible, elusive enemy, and the measures were unprecedented, but circumstances demanded them. (BBC news, 2020)

The statement “infectious disease control is fundamentally different “supports the analogy of President Macron. The disclosed system may be associated with fighting a pathogen poses the same challenge as fighting an enemy in war. Therefore, anyone involved in the fight against pathogens must have the same skills, mindset, and spirit as a General at war.

In the context of zoonotic diseases, the disclosed system may be based on these five statements as follows.

1. There are three ways that a nation gets what they want; planning, working, and truthfulness. Any great infectious diseases prevention operation takes careful planning or thinking. Then you must have a well-trained emergency organization and support of the population to carry it out: that's working. But between the plan and the operation, there is always truthfulness. That truthfulness spells defeat or victory, success or failure. It is the reaction of the people to the ordeal when it comes. Some people call that getting the breaks; I call it to hope. Hope has His part or margin in everything, that's where common sense comes in.

2. Sure, we want the current zoonotic measures to end. We want to End this fight against the pathogen. The quickest way to End this fight against the pathogen is to go and eliminate the pathogen which started it.

3. There is only one tactical principle that is not subject to change. It is to us breaking the chain of transmission to inflict the maximum amount of destruction on the pathogen in the minimum amount of time.

4. Now if you are going to win any battle you have to do one thing. You have to make the science run the politics. Never let the politicians tell the scientists what to do. The politicians may always give up. They are always tired morning, noon, and night. But the politicians are never tired if the scientists are not tired. When you were younger science could make you study all night, and the body was never tired. You've always got to make the scientist take over and keep combating the pathogen.

5. Keep combating the pathogen, someone must be on top, why not the secretary of Health and Human Services (HHS)?

The Public Health Service Act (PHSA)1 provides the legal authority for the Department of Health and Human Services (HHS), among other things, to respond to public health emergencies. The act authorizes the HHS secretary to lead federal public health and medical response to public health emergencies, determine that a public health emergency exists, and assist states in their response activities. This fact sheet focuses on Section 319 of the PHSA, which governs public health emergencies. (Public Health Service Act, Section 319)

Section 319 authorizes the secretary to determine that a public health emergency exists. This determination triggers emergency powers that permit the federal government to engage in activities such as assisting state and local governments, suspending or modifying certain legal requirements, and expending available funds to address the public health emergency. (Public Health Service Act, Section 319)

Section 319 authorizes the secretary to determine that a public health emergency exists if the secretary determines that a disease or disorder presents a public health emergency or that a public health emergency—including significant outbreaks of infectious diseases or bioterrorist attacks—otherwise exists. The secretary's action is specifically referred to as a “determination” in Section 319 and documents issued by HHS instead of the term “declaration.” The secretary may determine that a disease or condition presents a public health emergency based on conditions existing before an outbreak or natural disaster occurs.3 Although a Section 319 determination activates certain federal emergency powers, a public health emergency determination is not required for HHS to assist states; the secretary has broad legal authority under other sections of the PHSA to help states and localities. PHSA Section 301 gives the secretary broad authority to provide assistance and conduct investigations, among other things, into the causes, prevention, and control of diseases.1 PHSA Section 311 permits the secretary to work with states and localities to prevent and control communicable diseases and to implement plans activating HHS resources to control diseases and other public health emergencies.[1] The secretary may activate the National Disaster Medical System and deploy the Strategic National Stockpile without a Section 319 determination. (Public Health Service Act, Section 319)

Once the HHS secretary determines that a public health emergency exists, the secretary is authorized, consistent with the secretary's other authorities, to respond to the public health emergency. These authorities include making grants, contracting, and investigating the cause, treatment, or prevention of the disease or disorder underlying the public health emergency. The secretary may use funds from the Public Health Emergency Fund when funds are appropriated for it.

Over time Congress has added other authorities to the PHSA, the Social Security Act (SSA), the Federal Food, Drug, and Cosmetic Act (FFDCA), and other laws administered by the secretary that allows the secretary to make certain discretionary actions once a public health emergency is determined under Section 319. Other discretionary actions include: Using SSA Section 1135 to waive or modify certain requirements under Medicare, Medicaid, the Children's Health Insurance Program (CHIP), and the Health Insurance Portability and Accountability Act (HIPAA).
Exempting a person from select agent requirements for 30 days.
Waiving certain prescription and dispensing requirements under the FFDCA.
Adjusting Medicare reimbursement for certain Part B drugs.
Waiving certain Ryan White HIV/AIDS grant program requirements.
Making temporary personnel appointments for up to one year.
Granting extensions or waiving sanctions relating to the submission of data or reports.3

Duration A Section 319 determination remains in effect for 90 days or until the Secretary determines that the emergency no longer exists, whichever occurs first. If the same or additional conditions continue to warrant a public health emergency, the secretary may renew the determination for additional 90-day periods.

Stafford Act and National Emergencies Act—A public health emergency determination under Section 319 of the PHSA is separate and distinct from a presidential declaration of emergency under the National Emergencies Act or a Stafford Act declaration of an emergency or disaster. The HHS secretary does not need a presidential declaration to issue a public health emergency determination under Section 319. Unlike a Stafford Act declaration, which must be initiated by a gubernatorial request to the president, the secretary may make a Section 319 public health emergency determination without a state request. Declarations under other laws are required for the secretary to exercise certain authorities. For the secretary to exercise waiver authority under SSA Section 1135, there must be an existing public health emergency declaration in addition to a presidential declaration under the Stafford Act or the National Emergencies Act.

PREP Act—The secretary does not need to make a public health emergency determination under PHSA Section 319 to issue a declaration under the Public Readiness and Emergency Preparedness Act (PREP Act) for the liability immunity under the PREP Act to go into effect. The PREP Act, among other things, allows the secretary to provide tort liability immunity (except for willful misconduct) for claims of loss arising from the development and administration of countermeasures against diseases or threats that constitute a current or future risk of a public health emergency.

Section 319 does not contain any immunity provisions or liability protections. The determination of a public health emergency, however, is a prerequisite for the activation of liability provisions in other laws such as the PREP Act.

A determination by the HHS secretary under Section 319 that a public health emergency exists activates the secretary's emergency powers to provide states with resources and personnel to respond to the emergency and to waive or modify certain legal requirements. There is no statutory or regulatory requirement that a state submits a formal request to the secretary for the determination of a public health emergency under PHSA Section 319 or an SSA Section 1135 waiver to be made. HHS encourages states to work with regional emergency coordinators in HHS regional offices to facilitate the request if the state believes such determinations are needed to respond to an event. [3] A state declaration of emergency does not affect the secretary's Section 319 authority to determine that a public health emergency exists. However, the fact that a state has declared an emergency or requested federal assistance in response to an emergency may be relevant to the secretary's consideration of whether a public health emergency exists. (Public Health Service Act, Section 319)

The Pandemic Intervals Framework (PIF) describes the progression of an influenza pandemic using six intervals. This framework is used to guide influenza pandemic planning and provides recommendations for risk assessment, decision-making, and action in the United States. These intervals provide a common method to describe a pandemic activity that may inform public health actions. The duration of each pandemic interval might vary depending on the characteristics of the virus and the public health response. (Pandemic Intervals Framework)

Further, a description of the Six Pandemic Intervals may be as follows:

1) Investigation of Cases of Novel Influenza A Virus Infection in Humans

When novel influenza A viruses are identified in people, public health actions focus on targeted monitoring and investigation. This may trigger a risk assessment of that virus with the Influenza Risk Assessment Tool (IRAT), which is used to evaluate if the virus has the potential to cause a pandemic.

2) Recognition of Increased Potential for Ongoing Transmission of a Novel Influenza A Virus

When increasing numbers of human cases of novel influenza A illness are identified and the virus has the potential to spread from person to person, public health actions focus on control of the outbreak, including treatment of sick persons.

3) Initiation of a Pandemic Wave

A pandemic occurs when people are easily infected with a novel influenza A virus that may spread in a sustained manner from person to person.

4) Acceleration of a Pandemic Wave

The acceleration (or “speeding up”) is the upward epidemiological curve as the new virus infects susceptible people. Public health actions at this time may focus on the use of appropriate non-pharmaceutical interventions in the community (e.g. school and child-care facility closures, social distancing), as well the use of medications (e.g. antivirals) and vaccines, if available. These actions combined may reduce the spread of the disease, and prevent illness or death.

5) Deceleration of a Pandemic Wave

The deceleration (or “slowing down”) happens when pandemic influenza cases consistently decrease in the United States. Public health actions include continued vaccination, monitoring of pandemic influenza A virus circulation and illness, and reducing the use of non-pharmaceutical interventions in the community (e.g. school closures).

6) Preparation for Future Pandemic Waves

When pandemic influenza has subsided, public health actions include continued monitoring of pandemic influenza A virus activity and preparing for potential additional waves of infection. It is possible that a 2nd pandemic wave could have higher severity than the initial wave. An influenza pandemic is declared ended when enough data shows that the influenza virus, worldwide, is similar to a seasonal influenza virus in how it spreads and the severity of the illness it may cause.

In addition to describing the progression of a pandemic, certain indicators and assessments are used to define when one interval moves into another. CDC uses two tools (the Influenza Risk Assessment Tool and the Pandemic Severity Assessment Framework) to evaluate the pandemic risk that a new influenza A virus may pose. The results from both of these assessments are used to guide federal, state, and local public health decisions.

### (Pandemic Intervals Framework)

Further, the disclosed system may either prevent or treat the infection. Further, the disclosed system may facilitate preventing Infection Transmission (Breaking the Chain)

To manage and prevent the spread of infectious diseases, it is important to interrupt the chain of infection at any of the six links.

The specific preventative measures used may depend on the pathogen and its mode of transmission, however, the following list offers some suggested strategies for breaking the chain of infection at each link.

Infectious agents may still be present even if preventative measures are taken. However, the more measures implemented, the easier it may be to interrupt links of the chain. Prevention is most effective when a combination of measures is used.
Infectious agent: Diagnosis and treatment; and Antimicrobial stewardship.
Reservoir: Cleaning, disinfection, and sterilization; Infection prevention policies; and Pest control.
Portal of exit: Hand hygiene; PPE; Control of aerosols and splatter; Respiratory etiquette; and Waste disposal.
Mode of transmission: Hand hygiene; PPE; Food safety; Cleaning, disinfection, and sterilization; and Isolation.
Portal of entry: Hand hygiene; PPE; Personal hygiene; First aid; and Removal of catheter and tubes.
Susceptible host: Immunizations; Treatment of underlying conditions; and Patient education. (Ausmed Editorial Team, 2020)(37)

The Secretary of Health has to provide resources for all measures to prevent the preparation of infectious diseases. He faces the basic economic problem as follows:
The fundamental economic problem is the issue of scarcity and how best to produce and distribute these scarce resources.
Scarcity means there is a finite supply of goods and raw materials.
Finite resources mean they are limited and may run out.
Unlimited wants to mean that there is no end to the number of goods and services people would like to consume.
Because of unlimited wants—People would like to consume more than it is possible to produce (scarcity)
Fundamental economic question

Therefore because of scarcity, economics is concerned with:
What to produce?
How to produce?
For whom?

(Basic Economic Problem)

There is only one tactical principle that is not subject to change. It is to use the means at hand to inflict the maximum amount of wound, death, and destruction on the enemy in the minimum amount of time. (Patton)

Attack rapidly, ruthlessly, viciously, without rest, however tired and hungry you may be, the enemy may be more tired, more hungry. (Patton)

In this context, “Attack rapidly” and “the enemy in the minimum amount of time” mean to order a measure once the infectious disease is discovered. The only measure that meets this requirement for all future pathogens is isolation. At this time, the epidemiologist might not know the values for the incubation period, latent period, and period of communicability. Nevertheless, he may estimate to slow down the spreading of the disease. 85 to 95 percent of the population follow effective and efficient measures to prevent the spreading of infectious diseases

Debt monetization or monetary financing is the practice of a government borrowing money from the central bank to finance public spending instead of selling bonds to the private sector or raising taxes. It is often informally and pejoratively called printing money or money creation. This practice is generally prohibited in many countries because it is considered dangerous due to the risk of creating runaway inflation. However current central bank policies in responses to the COVID-19 pandemic (such as quantitative easing) are considered to be very similar to debt monetization, although with subtle and important nuances. (Debt monetization)

Economic uncertainty implies the future outlook for the economy is unpredictable. When people talk of economic uncertainty, they usually imply there is a high likelihood of negative economic events. Economic uncertainty could involve.
Predictions of a higher and more volatile inflation rate. (inflation uncertainty)
Concerns over economic downturn—lower economic growth or full-blown recession (negative economic growth)
People fear the prospect of being made unemployed.
Concerns over prospects for exchange rate—e.g. rapid devaluation of the currency.
Concerns over government borrowing—e.g. markets unwilling to finance more debt, leading to default.
Major change in economic structure

(Tejvan Pettinger, 2016)(47)

The current situation demonstrates that the most effective Covid-19 measures do not eliminate the COVID-19 disease.

If HHS secretary allows the current situation to continue, government borrowing may increase leading to markets being unwilling to finance more debt, leading to monetize debt, raising taxes, or default.

85% to 95% of the population voluntarily use the solution because they are doing everything, they may combat the pathogen. Where do the population get the courage to accomplish this impossible mission? The population mindset is war, so the population follows the knowledge of General Patton's statements to succeed. 85% to 95% of the population must have the desire to become achievers. In the sense of General Patton, achievers are necessary to take up the fight with the pathogens victorious.

As the politicians imagine, it may not suffice to say that we want every citizen to become an achiever. From not being an achiever to becoming an achiever is a change of behavior. A behavior change requires organizational change management. Companies invest millions of USD to get their employees used to the new process. Why should this be different for a government?

Organization And Change Management Consulting Market may reach USD 2 billion by 2024|SpendEdge (SpendEdge, 2021)

Governments change the behavior of the people through legislation. In the case of the elimination of disease, this concept does not work. How do governments force people to visit a doctor and punish them when they are sick? The person may say he did not feel sick. Therefore, he did not find it necessary to see a doctor.

The HHS secretary must have a project manager and a change manager for the elimination of the disease. The request of the Change Manager to the Project Manager is the following:

A growing body of data shows a strong correlation between the success of a change initiative and how well the people side is managed. Change projects with excellent change management are six times more likely to meet objectives and outcomes. (Core Roles in Change Management)(41)

The HHS secretary has the following perspective on the disease elimination project If he lacks the insight of change management.

My focus is getting to go-live. Once I flip the switch, I'm moving on to the next project.” (Core Roles in Change Management) (41)

Over 71% of Americans said they don't plan to download and use a COVID contact tracing app. Of those who said they would not use the apps, the main reason was concerns that the technology won't protect people's digital privacy. The most trusted COVID contact tracing app technology is that from Big Tech: 32% said they would trust technology by Google/Apple to keep their data secure and private. Just 14% said the same for contact tracing apps provided directly by the government. Further, those working in Government and Healthcare are the least likely to download the technology—84% of people from these sectors said they wouldn't use the apps. People age 25-44 rank COVID contact tracing apps as the biggest threat to digital privacy in 2020. They view the apps as a bigger threat than identity theft or even cybercrime. The least likely to use the apps are people over 55-88% said they don't intend on downloading the technology. Women are also far less likely than men to use the apps; only 18% of women say they would download the apps, while 40% of men said they would. Roughly 75% of Americans believe their digital privacy is at risk if COVID contact tracing app data is stored so government and authorities may access the data. (Avira, 2020)

Hesitancy comes from many sources. Common concerns include how quickly the COVID vaccines were developed and if they are safe. Many people have adopted a “wait and see” attitude in response to these concerns. Others have technical questions including if they should be vaccinated if they've already had COVID if the vaccines may be effective against different strains, and how we may know if they're safe for children.

Of course, in some cases, these questions are accompanied by a mistrust of medicine, public health, and government more generally. In communities of color, we may trace this mistrust back to a history of neglect and exploitation at the hands of health care providers and government, as well as people's recent and personal experiences with structural inequities in public health, medicine, and social services.

Because different types of hesitancy exist, it is important that any vaccine communication initiative is based on the concerns of particular communities and that they are sensitive to the underlying reasons for these concerns. (Megan Lowry, 2021)(42)

The gonorrhea control program described by Potterat et al. is a valuable model of the use of public health enforcement powers. Further, the gonorrhea control program may be a powerful cautionary tale for the future of effective public health enforcement in the United States. The key to the success of this program was a staff with expertise in disease control, a health director committed to disease control, effective legal support from the District Attorney's office, and the political support of the community. A disease control program that includes personal restrictions needs to meet these standards to survive a legal challenge. It must:

1. Address a real problem that poses a direct threat to third parties;

2. Develop a scientific control strategy;

3. Implement that strategy in the most effective way, with the least restrictions consistent with the resources available;

4. Evaluate the program periodically to show that it is working; and

5. Phase out the program when it is no longer epidemiologically sound.

Except in very unusual circumstances, every one of these requirements involves a value judgment made on a continuum. Public health seldom has clear and simple answers. Even wondrously effective strategies such as childhood immunizations become problematic when they have achieved such great success that the risks of the immunization exceed the residual risk of the disease. The courts never demand perfection in public health, nor do they demand the least restrictive approaches if those are not compatible with the resources available to the program or with minimizing the risk to third parties (Richards & Rathbun, 1999).

To achieve 85% to 95% usage of the solution, the best protection of the private sphere is mandatory. The best technical solution is not to use personal data, centralized data storage, and use Isolation to combat the pathogen. The best organizational solution for success depends highly on staff with expertise in disease control, an HHS secretary committed to disease control, effective legal support from the District Attorney's office, and the political support of the community. The HHS secretary secures popular support through the change process.

Further, designing the business process may include occurring of two demands simultaneously. Further, a first demand of the two demands may include hazard-generated demands that may include warning, pre-impact preparations, search and rescue, care of injured and dead, welfare needs, restoration of essential services, protection against continuing threats, and community order. Further, a second demand of the two demands may include response-generated demands that may include communications, continuing assessment of situation, mobilization, and utilization of resources, coordination, and exercise of authority. Emergency Management and the Incident Command System (44)

| Incident | | | |
|---|---|---|---|
| Response | Pandemic Response | Tool | In Charge |
| Warning | Investigation | IRAT | Secretary |
| | | | of HSS |
| Pre-Impact | Collect all | PReT | Safety Officer |
| preparations | encounters | | |
| Search and | Separate the contacts | PReT | Incident |
| rescue | from non-contacts | | Commander |
| | and place the | | |
| | contacts in | | |
| | quarantine | | |
| Care of | Medical and | PreT | Incident |
| injured and | psychologic | | Commander |
| dead | Treatment of | | |
| | Contacts and | | |
| | Infected Persons | | |
| Welfare needs | Solving the daily | PreT | Incident |
| | problems of the | | Commander |
| | Contacts and | | |
| | infected Persons | | |
| Restoration | Discharge the | PreT | Safety Officer |
| of essential | contact and sick | | |
| services | | | |
| Protection against | Preparation | IRAT | Secretary |
| continuing threat | | | of HSS |
| Community order | Health Hold Orders | Out of scope | District |
| | for Disease Control | of this paper | Attorney's |
| | | | office |

The enemy is not the population. The fight is not against the people but the pathogen. Therefore, ZooReS, an exemplary embodiment of the disclosed system herein, may collect information about the pathogen and not about the people.

Further, in an instance, a person knows which wristwatch he owns. The wristwatch informs the user about the time without any personal data. If the wristwatch is lost, the finder cannot find out the owner because there is no personal data on the watch. “ZooReS” uses this concept. It collects data about the persons wearing the device. As long as the wristwatch wearer does not change watches with other persons, the data integrity is guaranteed. It is not in the interest of the wearer of his wristwatch to swap with another wearer of a wristwatch. He may be quarantined because of the exchange and not because of infecting, thus restricting his movement space.

Further, for protecting personal data, a first principle may be the results of a query must be inherent anonymous. There must not be any conversion method from private data to anonymous data in the processing chain. The result contains only information about the transmission.

Civil Legal Organizations, Universities, and “anti-uncle Sam is watching you” Organizations must ensure compliance with these principles. The third principle is the transmission tracer cannot down-load or up-load data or program. The technical implementation of this principle requires decentralized data storage and the program area is a read-only memory.

Further, the disclosed system may be associated with decentralized data storage. The pathogen reproduces decentral and not central.

The carriers of the transmission tracer may undermine the concept by exploiting the second principle. They may exchange transmission tracers among themselves. Although the person must be foolish, this abuse is possible. Why is the person stupid? He risks sitting in quarantine for another person. Anyway, a personal assignment of the transmission tracer does not help. These persons exchange their transmission tracers only for a short time, and after that, they return them to the right person. A person with bad intentions and appropriate means may defeat any logic.

There is a great deal of talk about loyalty from the bottom to the top. Loyalty from the top down is even more necessary and much less prevalent. One of the most frequently noted characteristics of great men who have remained great is loyalty to their subordinates. (Patton).

Further, a Transmission Tracer (TraT) associated with the disclosed system records all possible sub-types of transmissions. The sub-types are touching, sexual intercourse, biting, direct projection of droplets, Across the placenta, and Airborne. The sub-types are irrelevant for the control of the disease. The maximum range for transmission of infection is significant. The epidemiologist determines the value.

The radio wave contains a unique recognition designation of the “TraT” and separation. The recognition identifier may be any string with a bijective mapping to the set of a telephone number. The data overhead is minimal when using the E.164 international standard, a telephone number with 15 digits. The separation is binary, true or false. True means quarantine or isolation. The absence of quarantine and isolation is a consequence of principles one and two. A person with a sniffer reads the message knows as much as with his own “TraT.” Someone with the separation value “True” may be either contaminated or a contact. The person with the sniffer gained no health information about the person wearing the “TraT”. If a person learns that someone is in quarantine, then it is the person's fault in quarantine. If the person had remained in quarantine, no one would know this fact.

Further, the disclosed system may facilitate breaking the transmission chain. [Concept of Incubation Period (period, 2020) (14)]

Further, a carrier “Patient Nr 0” may be contaminated and wears “TraT” having unique recognition designation “Nr.0”. The Transmission Chain Breaker (TCB) requests this “Trat of Nr.0” to set the separation value to True. This request includes the values of Beginning date of Incubation; Incubation period, Latent period, and Period of communicability according to the “Concept of Incubation period”.

Further, the Trat of Nr.0 sets the separation value to true and calculates the end date of the period of communicability using the beginning date. It selects all transmissions “Comun of Patient Nr.0” having a date within the period of communicability. The selected transmissions (Contact A) consist of the date of the transmission and the cellular telephone number of the TraT. The selected transmission is the set of cellular telephone numbers labeled “TraT of Contacts A”. The “TraT of Contact of Nr.0” sends a message to TraT “Trat of Contact A” using their cellular telephone numbers. The message requests the “TraT of Contacts A” to set its separation to true.

LABEL A: The message also contains the values of the incubation period, latent period, period of communicability, and date of transmission. The requested TraT of Contacts A calculates the end date of the incubation period. If the current date is within the incubation period and not within the latent period, then the TraT of Contact A sets the separation to true else it waits until the current date is not with the latent period.

Regardless of the separation value, the requested TraT of Contact A may calculate the start and end date of the period of communicability. “Comun of Contract A” represents the period of communicability in the diagram. The “Trat of Contact A” selects all transmissions having a date within the period of communicability. “Contacts B” represents the selected transmission of Contact A due to the period of communicability. The selected transmissions consist of the date of the transmission and the cellular telephone of the Trat of Contact B. The Trat of Contact A sends a message to TraT of Contact B using the cellular telephone number. The message requests the TraT of Contact B″ to set its separation to true. The procedure continues at LABEL A.

Further, the disclosed system may facilitate ensuring the enforcement of the quarantine measures. A TraT with the separation value “True” shares this value with the other TraT during transmission. The other TraT alert the carrier about the danger. The alerted carriers may spread out in all directions to comply with social distancing. If the carriers do not keep the social distancing for a specific duration, the Tract sets their separation values to True without causing a chain reaction.

Further, the Trat informs the carrier about another carrier who lives in a border region or is cross-border. Further, the TCB requests a TraT of a contaminated Person to attempt to find the corresponding Patient Nr. 0. In this case, the TCB transmits the following data to the TraT as follows: incubation period, latent period, period of communicability, the cellular telephone of the contaminated person, the end date of the incubation period, a set of at least 3 telephone numbers of contaminated person, and n number defining the depth of the tree.

The TraT of the contaminated person calculates the start date of the incubation based on the end date of the incubation time. It utilizes the start date of the incubation time as the End date of the Communicability period. LABEL B: This Tract uses the End date of the communicability period to calculate the beginning date of the communicability period. It searches for all transmissions between this start date and the End date of the communicability period. susceptibility A respectively B represent all the transmissions between this start date and End date of the communicability period transmission in the diagram. This TraT respectively susceptibility A reduces n by one and request from the tracts of susceptibility A respectively susceptibility B to find the Patient Nr. 0. The TraT of susceptibility A respectively susceptibility B check the value of n. If n is greater than zero then the TraT of susceptibility A respectively susceptibility B proceed to LABEL B.

If the value of n is zero, which is the case for susceptibility B in the diagram. Further, the TraT of susceptibility B constructs their tree structure. They search in their tree structure for nodes with the cellular telephone number of the provided set of a contaminated person.

If the search is successful, then this TraT transmits the following data to the TCB: a cellular telephone number of the Patient Nr.0 and a cellular telephone Number of the contaminated Person.

The TCB request this computation for every contaminated person.

The TCB creates tasks with the required values and sends these tasks to the Trats. It is the only device to communicate with the TraT. A TraT may communicate with other TraT only through the TCB.

The TCB manages the backtracking process.

The place of the quarantine guard is in front of the entrances of buildings. If a person without TraT approaches the gate, then the quarantine monitor alerts the doorman. The doorman makes sure that this person does not enter the building.

The quarantine monitor has a camera. It checks if a TraT sends its telephone number and separation value as soon as it detects a person. If the QG receives no signals, then a message is sent to the doorman. If the QM gets a signal, but the separation has the value True, a note goes to the doorman.

Further, the disclosed system may facilitate preventing the spread of infectious diseases.

Once an infectious agent leaves a reservoir, it must get transmitted to a new host if it is to multiply and cause disease. The route by which an infectious agent is transmitted from a reservoir to another host is called the mode of transmission. It is important to identify different modes of transmission because prevention and control measures differ depending on the type.

Further, mode of transmission may include direct mode of transmission associated with sub-types of transmission comprising touching, sexual intercourse, biting, direct projection of droplets, across the placenta. Further, the mode of transmission may include indirect mode of transmission associated with sub-types of transmission comprising airborne, vehicle-borne, and vector-borne

Summary of Different Modes of Transmission. (Modes of Transmission)

Direct transmission refers to the transfer of an infectious agent from an infected host to a new host, without the need for intermediates such as air, food, water, or other animals. Direct modes of transmission may occur in two main ways:

Person to person: The infectious agent is spread by direct contact between people through touching, biting, kissing, sexual intercourse, or direct projection of respiratory droplets into another person's nose or mouth during coughing, sneezing or talking. A familiar example is the transmission of HIV from an infected person to others through sexual intercourse. (Modes of transmission)

Transplacental transmission: This refers to the transmission of an infectious agent from a pregnant woman to her fetus through the placenta. An example is mother-to-child transmission (MTCT) of HIV. (Modes of transmission)

Indirect transmission is when infectious agents are transmitted to new hosts through intermediates such as air, food, water, objects or substances in the environment, or other animals. Indirect transmission has three subtypes:

Airborne transmission: The infectious agent may be transmitted in dried secretions from the respiratory tract, which may remain suspended in the air for some time. For example, the infectious agent causing tuberculosis may enter a new host through airborne transmission.

Vehicle-borne transmission: A vehicle is any non-living substance or object that may be contaminated by an infectious agent, which then transmits it to a new host. Contamination refers to the presence of an infectious agent in or on the vehicle.

Vector-borne transmission: A vector is an organism, usually an arthropod, which transmits an infectious agent to a new host. Arthropods that act as vectors include houseflies, mosquitoes, lice, and ticks. (Modes of transmission) (27)

The elimination strategy aims to eradicate the pathogen from an area. How does the country prevent the invasion of new pathogens from the surrounding countries? People who are in the country for a specific time, quarantine themselves before arriving. The place of quarantine would be a hotel. The hotel ensures compliance and journey to the transport vehicle.

The problem is the border crossers. The problem is solved when all neighboring states also have a “ZooReS in use. Until then, the inhabitants may have to be satisfied with an interim solution. The interim solution distributes a TraT to all cross-border commuters. The TraT may quarantine residents of non-border areas as soon as they are in the range of the radio wave of border crosser, or a resident of a border area and have exceeded a maximum duration.

The inhabitant of the decontaminated area remains in quarantine by the amount of the difference of incubation period minus latent period. Further, life becomes predictable and controllable for the resident. The controllability and plan ability of life for the inhabitants is what this development wants to achieve. You no longer have to fear that a politician may exercise his power and thus cross your plans.

The TraT maps the “subtype of the transmission” as the signal strength of the radio wave and duration time. The different subs types have different ranges or different duration times. However, since it is not known which subtype a future unknown pathogen may use, the best choice is to use the distance for a “Direct projection of droplets.” This distance is the distance for social distancing.

Assuming that each TraT has ten transmissions per day, that the spread of the disease corresponds to a tree structure with the node being TraTs, and that each inhabitant of the earth carries a TraZ, the question arises whether the patient zero is not a computable number.

An algorithm associated with the disclosed system follows the descendant (Tracts) to level 10. There are 10¹⁰ children at this level. The algorithm stops at this point because the number of children cannot exceed the world pollution of approximately 8*10⁹.

So, a tree may exist with threads of 10¹⁰ people leading to the contaminated person. Which of these 10¹⁰ people is the patient zero? The Algorithm creates a tree for each person based on his TraT up to the ten levels. It searches in this tree for the phone numbers of the other contaminated persons. The root of the tree with the phone numbers of the contaminated person is the patient zero. So this Algorithm has a time complexity of 10¹⁰*10¹⁰=10²⁰. This Algorithm is not computable. There are 22*10⁹ SMS per day worldwide. The ZooReS needs the SMS infrastructure.

The description of the Pandemic level 1 suggests that this point of time is consistent with the point of time of the WHO pandemic phase 2. The WHO describes the pandemic phase 2 as follows:

An animal influenza virus circulating in domesticated or wild animals is known to have caused infection in humans and is therefore considered a specific potential pandemic threat. (Who Pandemic phase Descriptions and Main Action by Phase) (44)

The WHO has planned the following actions for this phase for communication and to reduce the spread of disease:

Complete communications planning and initiate communications activities to communicate real and potential risks. (Who Pandemic phase Descriptions and Main Action by Phase) (44)

Promote beneficial behaviors in individuals for self-protection. Plan for use of pharmaceuticals and vaccines. (Who Pandemic phase Descriptions and Main Action by Phase) (44)

As soon as the security officer knows a location pattern, personal characteristic, or TraT Telephone number in relationship to contamination, the ZooReS is ready for use. With this information, the success of the combat depends on the incident command system (ICS) set up and operated by the government.

The ICS guides how to organize assets to respond to an incident (system description) and processes to manage the response through its successive stages (concept of operations). All response assets are organized into five functional[10] areas: Command, Operations, Planning, Logistics, and Administration/Finance.

The ICS, as described in NEVIS, refers to the combination of facilities, equipment, personnel, procedures, and communications operating within a common organizational structure and designed to aid in the management of resources during incident response. The ICS is based on eight concepts that contribute to the successful application of this system

The activity of “preparing for” belonging to the ICS has prepared the population for this situation. The Public information official urges his population who fit the pattern to see their family doctor. The family doctor examines according to the guidelines of the health authorities. The physicians note the TraT phone number and date of the positive result and send this information to the TCB. The safety officer requests the TCB to set the separation value of the TraTs of the entered telephone number to true.

Further, the disclosed system may heuristically Approach to breaking the Transmission Chain.

US residents send 6 billion texts per day. Further, the SMS infrastructure of the USA has 10 percent reserves. Therefore, the ZooReS may generate 600 million SMS without the SMS infrastructure collapsing. In addition, a trace in a tree trunk on two levels generates 10,000 messages. The 10000 messages per potential patient zero allow to process 60 000 cases per day. This number corresponds to the peak of COVID-19 in August 2020 in the United States.

It would have been possible to spare the USA from the wave in November 2020. Had a close collaborator of the state secretary of HHS, defense, economy, or budget had this idea of the ZooReS and convinced the state secretary to follow it in January 2020. With its inexhaustible technical and organizational capabilities, the USA could have developed and distributed the ZooRes in 7 to 9 months. Had this been the case, today the USA would be Covid-19 free like other diseases like malaria, yellow fever, smallpox, etc.

The US SMS infrastructure may still process the 250,000 positive cases per day. ZooReS generates 2.5 billion messages. This number is still smaller than the current 6 billion messages per day. So, if the population generates ever second messages during a public health emergency, there would be enough capacity.

The TCB puts theoretical 2.5 billion people in quarantine per day at 250,000 positive cases per day. At this rate of countermeasure, the entire US population would be in quarantine within 1 day, which is equally significant to a lock-down. A lock-down leads to breaking the transmission chain. Therefore, it follows that the algorithm leads to breaking the chain of transmission. In reality, this number is much smaller because many telephone numbers are redundant.

Further, the disclosed system may be associated with an algorithm for breaking the Transmission chain.

Once the security officer enters the values of the concept of Incubation into the TCB, the TCB selects the oldest positive case in the TCB. The TCB uses the phone number of the oldest case to compute a potential patient zero according to the algorithm. The TraTs stop at two levels of the tree structure. Thus, 100 possible patient zeros are possible if each node contains ten phone numbers. For each of the 100 likely “patients zero”, a level 2 tree structure is created. In these tree structures, the TraTs search for the phone number that corresponds to the positive cases.

The root of the tree with the most positive cases is the selected patient zero. The TCB requests the TraT with the Telephone number of the select patient zero to compute the corresponding contacts. All Trats set their separation value to true for those contacts fulling the concept of incubation at the given current time. The TCB searches for the next oldest positive case not mapped to a tree structure. A successful search attempt causes the algorithm to start with the found telephone number of the positive case; otherwise, the algorithm stops.

Further, in an instance, the incubation period is 14 days, the latency is 13 days, and the period of communicability is one day. Patient 0 lives in a country without ZooReS. Patient 0 contaminated patients 1 and 1* while they were abroad. Patients 1,2,4, 1*, 2* and 10 are asymptotic cases. On June 14, 20xx, the primary care physician reports case 5. The TCB requests Trat of patient 5 to set the separation to true. The Trat of patient 15 requests TraT of patient 15 to set his separation to true.

On June 28, 20xx, the primary care physicians report cases 8 and 3*. Patient 8's reporting causes the TCB to set the separation value to true for the Trats of patients 12 and 13. Patient 8's TraT may find transmission 7 and 2 for June 14, 20xx. It instructs the TraTs of patients 2 and 7 to find the pseudo patient zero. The TraT of patient seven may find any ten TraTs. The TraTs belong to patients 20 to 30. It is unlikely that patient five may occur when generating the trees of patients 20 to 30. Therefore, patients 20 to 30 are not considered pseudo patient 0. If Patient 5 appears in a tree structure that belongs to Patient 20 to 30, it is a false positive. A false positive does not prevent the break of the transmission chain.

The TraT of patient 2 finds the TraT of patient one and others as pseudo patient null. Person 31 to 40 are the other pseudo patient null. The TraTs of persons 31 to 40 generate their tree structures. Patient 5 should not appear in these structures. But in the tree structure of patient 1, patient 5 occurs. The TCB designates patient one as a pseudo patient and causes the TraT of patient 1 to set its separation value to 1.

Setting the separation value of TraT from patient 1 to true causes a chain reaction of TraTs. The TraT of patient one does not set its value to 1 because the infectious period is one day. Instead, the TraT of patient 1 instructs Trat of Patient 4 to set its separation value to true. TraT 4 rejects the request on the same accounts of Trat 1, but the TraT of patient 4 asks the TraT of patient 10 to set the separation value to 1. Patient 10's TraT complies with the request and also sets the separation value from 17 to true.

The algorithm continues with patient 3* following the same pattern as patient 8.

Further, the disclosed system may not be limited to zoonosis diseases. It may be applied to any infectious disease. The prerequisite is that the measures to break the transmission chains are known and feasible.

Further, indirect transmission does not contain a path to determine the contacts. Elimination is with the ZooReS is still possible. The TCB quarantines half of the population by setting the status of half of the TraT to quarantine. The essential operations are organized in such a way that half of the employees have to be in quarantine. The first half of the population may be quarantined in the first phase.

An indeterminate number of phase 1 people may get sick. People of phase 2 may take care of these people of phase 1. The TCB quarantines the phase 2 person after the incubation period of the phase 1 person. The phase 1 persons take care of the phase 2 patients under strike hygienic measures.

The TCB quarantines the person in increments of 10% or less. TraTs independently determine the number of people in the household. This is possible due to the function that quarantines the person if they are with a person in quarantine for too long.

Further, a textbook version of contact tracing starts with someone testing positive for COVID-19 and isolating themselves. Contact-tracer interviews this person to find out who they might have exposed while infected, usually from 48 hours before the positive test, or before symptoms appeared (if there were any). Close contacts—those who've spent more than 15 minutes close to the infected person—are of special interest, but anyone who shared public transport or office space might qualify. Tracers then call or visit those contacts to tell them they need to quarantine so that they don't pass the virus on to more people. The chain of transmission is broken.

In reality, failures occur at every stage of this test-trace-isolate sequence. People get COVID-19 and don't know it, or delay getting tested. Positive results may take days to be confirmed. Not everyone who tests positive isolates when requested; one survey in May found that in the United Kingdom, 61% of people who were self-isolating said they'd left their house in the past day. People can't always be reached for an interview or don't provide details of their close contacts. And not all contacts are reached or are willing to comply with quarantine orders.

Because of this series of problems, researchers estimate that in England this year, tracers typically reached less than half of the close contacts of people who′d had a positive COVID-19 test (see ‘Missed contacts’). There are no data on how many of these contacts were quarantined in turn (Lewis, 2020).

The United States is in a particularly dire situation. “Public-health authorities are struggling to reach cases and contacts” despite aggressive efforts, says John Oeltmann, head of contact tracing assessment at the US Centers for Disease Control and Prevention (CDC) in Atlanta, Ga. He and his team evaluated two counties in North Carolina. In June and July, 48% of cases in one county and 35% in the other reported no contacts. Of the contacts whose details were provided, one-quarter in one county and almost half in the other couldn't be reached on the phone after three attempts over consecutive days. In New Jersey, just 49% of cases between July and November were contacted; only 31% of those provided any contact details. “These results are not rare,” says Oeltmann. Such data, which demonstrate how poorly contact tracing is working, are scarce. Only a handful of US states openly report contact-tracing metrics. And although the European Centre for Disease Prevention and Control (ECDC) lists the types of data that countries should gather to monitor contact-tracing efforts, none is reported back to the ECDC or is readily available for comparison. This makes it difficult to compare failures in contact tracing between countries. (Lewis, 2020)

The concept of the ZooReS starts with someone seeing the doctor and the doctor faced with unknown symptoms or someone testing positive for a disease. The ZooReS does not interviews anyone to find out who they might have exposed while infected, usually from 48 hours before the positive test, or before symptoms appeared (if there were any). The TraT knows who the person might have been exposed to while affected. Close contacts—those who've spent more than 15 minutes close to the infected person—are of special interest, but anyone who shared public transport or office space might qualify. The TCB does not call or visit those contacts to tell them they need to quarantine so that they don't pass the disease on to more people. The TCB sends the contact an SMS Message to tell them they need to quarantine. The chain of transmission is broken.

In reality, the failures of contact tracer are theoretical in respect to ZooReS. The ZooReS circumvents the problem of people get a disease and don't know it, or delay getting tested. It does it by searching for the Patient Nr 0. There is always a path. transmission from patient Nr.0 to people get an infectious disease and not knowing it. The ZooReS concept does not intend to mitigate the disease but eliminate it. Therefore, it plays its save and relies on doctor observation which does not take days to be confirmed. Furthermore, it implemented at a stage where there are only very few cases daily. A positive tested person is known to all they encounter when they leave their home. Social control may make leaving the house unpleasant for contaminated persons.

The TCB and TraT do not rely on Information obtain from Interviews. The police force or national guard may be called upon to arrest people who do not comply with quarantine orders. The police officer's TraT may track down all those who are not willing to comply with quarantine orders.

Further, not all user are going to stay for their lifetime in quarantine. Only 2% of the user right here today would quarantine in a pandemic. Quarantine must not be feared. Quarantine, in time, comes to a selected few. Yes, every person dislike being locked up in his first quarantine. If he says he's not, he's a liar. Some persons are selfish but they quarantine the same as the unselfish or they get the hell slammed out of them being watched by unquarantined person who are just as scared as they are. The real hero is the person who might be a false positive even though he is locked up.

The ZooReS may communicate to the public that people's privacy is protected by not using any personal data and the use of the data collected is always controlled by the wearer of the TraT. Furthermore, the Americans do not have to trust the government for the success of this important venture, because the government is only involved in financing the development and the operation of ZooReS. The success of this important venture depends entirely on the Americans wearing the Tact, seeing their primary care physician, and abiding to quarantine.

The Doctors associations may operate the ZooReS Infrastructure. The TCB never stores the fetched data locally. Governments may still be tempted to link the TraT Id with the names of the person.

In case of such abuse, the persons may claim a violation of the social contracts and destroy their TraT. The destruction makes it impossible for the government to access the data. For this reason, every government may be careful not to break the social contract. A breach of the social contract would render the ZooReS useless because a majority of the population would destroy their TraTs.

10 to 15 percent of the population may reject the ZooReS for irrational reasons. These people must be forced by law to wear the devices or to go to quarantine.

A disease control program that includes personal restrictions needs to meet these standards to survive a legal challenge. It must address a real problem that poses a direct threat to third parties, develop a scientific control strategy, implement that strategy in the most effective way, with the least restrictions consistent with the resources available, evaluate the program periodically to show that it is working; and phase out the program when it is no longer epidemiologically sound. (Richards & Rathbun, 1999) (21)

This is a list of diseases known (or declared) to have been eliminated from the United States, either permanently or at one time. Most of the diseases listed were eliminated after coordinated public health campaigns. (Since some diseases may be eliminated and then reintroduced at a later time, such diseases are still eligible for the list, but with the fact of reintroduction noted.) Some entries are based on formal public health declarations, others are based on reliable information in the medical or public health literature. Since some diseases may be eliminated, but subsequently reimported without transmitting additional endemic cases, these are noted in a dedicated column. Although, no fixed rule always applies, many infectious diseases (e.g., measles) are considered eliminated when no cases have been reported to public health authorities for at least 12 months. (diseases eliminated from the United States, 2015) 44

Further, a coordinated campaign significantly improves the chance of the successful elimination of a disease. To such a campaign belongs the sub-campaign with the following goals for the ZooReS.

1. 85 to 95% of the Population wears the Trat.

2. 85 to 95% of the population see their primary physician

3. 85 to 95% of the population obey the call to stay in quarantine.

4. Point two is the Achilles heel of the ZooReS concept. Without the data of the positive cases, the TCB cannot send the message to go into quarantine. Thus, ZooReS may not break the transmission chain.

The goals of the campaign require the people to change their behavior. So besides project management, change management is essential for the successful operation of the ZooReS.

A growing body of data shows a strong correlation between the success of a change initiative and how well the people side is managed. Change projects with excellent change management are six times more likely to meet objectives and outcomes. (35)

The campaign may be associated with a planned group of especially political, business, or military activities that are intended to achieve a particular aim. (46) The change Management Process is based on Bruce Tuckmann's model for grouping development as a framework. Therefore, the planned group of activities are Forming, Storming, Norming, and Preforming. The Change Management Process is successful if 85 to 95% of the Population has this perspective of their role in eliminating the disease.

The team meets and learns about the opportunities and challenges, and then agrees on goals and begins to tackle the task. (Tuckman's stages of group development)

If the HHS secretary relies on the General's knowledge, this seemingly impossible task is accomplished with a fighting spirit.

The campaign team organizes rallies to discuss the opportunities and challenges of elimination. The group engages the mass media to explain the concerns about elimination. It presents the introduction of the TraT as its proposal to realize the elimination. The campaign team invites the Opponents of the government's proposal to submit a counter-proposal.

Storming may be the second stage of team development, where the group starts to sort itself out and gain each others' trust. (Tuckman's stages of group development) (4)

How should the people trust the government in dealing with the Covid-19 pandemic? Fake news is commonplace in this world. Scientific facts may be declared as fake news by opponents. These circumstances force a different approach. Convincing the population must be done in the form of a campaign similar to an election campaign. The campaign focuses on insecurity in society, its effects, and elimination. The campaign team encourages the population to guide their decision not by party affiliation, political affiliation, belief, sympathy to opponents, and antipathy to supporters, but by common sense.

Impact of COVID-19 on people's livelihoods, their health and our food systems Joint statement by ILO, FAO, IFAD, and WHO 13 Oct. 2020|Statement|Reading time: 3 min (864 words) The COVID-19 pandemic has led to a dramatic loss of human life worldwide and presents an unprecedented challenge to public health, food systems and the world of work. The economic and social disruption caused by the pandemic is devastating: tens of millions of people are at risk of falling into extreme poverty, while the number of undernourished people, currently estimated at nearly 690 million, could increase by up to 132 million by the end of the year. Millions of enterprises face an existential threat. Nearly half of the world's 3.3 billion global workforces are at risk of losing their livelihoods. Informal economy workers are particularly vulnerable because the majority lack social protection and access to quality health care and have lost access to productive assets. Without the means to earn an income during lockdowns, many are unable to feed themselves and their families. For most, no income means no food, or, at best, less food and less nutritious food. The pandemic has been affecting the entire food system and has laid bare its fragility. Border closures, trade restrictions, and confinement measures have been preventing farmers from accessing markets, including for buying inputs and selling their produce, and agricultural workers from harvesting crops, thus disrupting domestic and international food supply chains and reducing access to healthy, safe, and diverse diets. The pandemic has decimated jobs and placed millions of livelihoods at risk. As breadwinners lose jobs, fall ill, and die, the food security and nutrition of millions of women and men are under threat, with those in low-income countries, particularly the most marginalized populations, which include small-scale farmers and indigenous peoples, being hardest hit. 26.7.2021 Impact of COVID-19 on people's livelihoods, their health, and our food systems (Joint statement by ILO, FAO, IFAD, and WHO, 2020).

It is not important whether a part of the population does not believe in the effector in the existence of Covid-19. The fact is that there is increased uncertainty because Covid-19 measures are in place. The question is whether to increase or decrease the amount of the Covid-19 measures. If opponents want to increase the Covid-19 measures then the mitigation strategy is the best, otherwise the zero covid strategies. If opponents do not believe in the reduction of the Covid-19 measure by ZooReS then they should at least give it a chance to decrease the Covid-19 measures.

This Covid-19 measure exists because part of the population believes in the existence of Covid-19 and its effects. The opponents of these Covid-19 measures are doing themselves a favor by giving the ZooReS a chance. If it works, there may be fewer Covid-19 measures in force and if there are more than before, switch it off.

The recommendation of Cecile Philippe and Nicolas Marques in their paper “The Zero Covid strategy protects people and economies more effectively” should be followed. These recommendations are reformulated in a country-neutral way in this paper. In particular, point 5 is tailored for the ZooReS.

Recommendations may be as follow:

1. Invite an open dialog about the adoption of a Zero Covid strategy to include experts from countries that have implemented it thus far.

2. Rally the diplomatic network and parliamentarians representing national citizens abroad to broaden feedback on Zero Covid strategies.

3. Assign mandates to assess the advantages and disadvantages of strategies for fighting Infectious Decease by calling, in particular, upon national public organizations that analyses public policy (

4. Organize feedback from national communities that have implemented the Zero Covid approach.

5. Support pilot projects in parts of the nation when local executives are receptive to the advantages of the Zero Covid strategy.

6. Bring the Zero Covid strategy into an analysis of risks related to the health crisis at both the national and international levels. The WHO may be a major player in the strategy's coordination

By the way, the knowledge of the named generals is not nonsense. If the American politicians and generals had taken into account the statement of President Eisenhower 20 years ago, the Afghan catastrophe would not have been a surprise.

The HHS Secretary is using this campaign to raise public may to fight for the elimination of the pathogen. This may to fight is necessary to make the Elimination possible. The impossible is the participation of 85 to 95 percent participation of the population.

A prerequisite for successful elimination is 85 to 90 percent participation of the population in the program. So, 80 to 95 percent of the population must have the right to fight.

This campaign of the HHS Secretary may test the maturity of the population for an elimination of the pathogen. The maturity of the population results from the necessity of missing personnel data. The HHS secretary believes that 85 to 95 percent of the population are honest and sincere and may not deliberately do anything to harm the ZooReS-Concept. Honesty and sincerity coupled with the may of a nation to fight is the critical success factor for the ZooReS concept. The may to fight must be in the blood of a nation to defeat the pathogen.

Every nation may claim that it has the may to fight but no one else to my knowledge as claimed this character to a nation expect General Patton “Americans love to fight. All real Americans love the sting of battle”. This means elimination or eradication program is more likely to succeed in the USA than anywhere else.

A thousand words cannot explain better what may to fight is than the following two youtube videos. The first one is to watch the non-fighting-oriented herd of buffalo with the following link: https://www.youtube.com/watch?v=qLqLpczbOnQ and the second one is the fighting oriented herd of buffalo: https://www.youtube.com/watch?v=LU8DDYz68kM.

The fighting-oriented herd and nonfighting-oriented herd save their respective calf but have different lead characters. In the first video, it is unclear to the speaker what characterizes the lead animals. In the second video, the audience is impressed by the size of the lead buffalo. The may to fight the enemy of the lead buffalo and of the herd makes the impossible possible. Countries need leaders and inhabitants who have the may to fight the pathogen. The packs in the second video may not bother this fighting-oriented herd of buffalo anymore. The Elimination concept is bonded to a high degree to fail with leaders and people without a fighting spirit. The HHS secretary assures with representative surveys the readiness of the country to fight the pathogen. He tests the fighting spirit of his nation. Each buffalo took the fight to the lions. None of the buffalos stood aside and watch the others do the fighting. Nonmatter the herd in the non-fighting-oriented herd is much larger than the fighting-oriented herd the lack of fighting spirit made them scarifies a buffalo. They are going to scarify more buffalos because the pack may stay close as they made prey. They are managing the problem but not solving it. My version of a General Patton quote is the following

There are two types of successful persons. Those who get on getting on by managing the problem and those who by solving the problem. One has to choose a system and stick to it; people who are not themselves are nobody.

The combat-oriented approach to solving a problem is not easy. It requires the following characteristics of a leader and institution, namely, wisdom, courage, prudence, truthfulness, and generosity.

The lead buffalo display his wisdom by leaving the calf behind to get help and applying a divided and conquer strategy. Courage was a display by fighting the lions.

Prudence was a display by taking their time to organize themselves before staging the attack. Truthfulness was displayed by the strongest buffalos spearhead the attack. Generosity was displayed by every buffalo taking part in the attack. The reader may try and discover these characteristics with the non-combat-oriented herd.

The HHS secretary must believe in his country, in eliminating the disease, and in getting the opponent to participate. The challenge that this belief brings with it requires, in my opinion, a modus operandi. The non-fighting spirit persons lack this modus operandi and therefore are intimidated by the task ahead. Only people with this modus operandi of a fighting spirit may try to make the impossible possible. In my opinion, the modus operandi of General Patton was “Americans love a winner. Americans may not tolerate a loser”. (Patton) A modus operandi does not have a positive effect in every situation. This is the reason the general's legacy is controversial.

This modus operandi is responsible for the controversial and sometimes erratic behavior but also for martial prowess. Modus operandi may come across as arrogant, smug, racist, nationalistic, etc. The important thing is to know your modus operandi, but not to let other people feel the negative aspects.

Especially the friends of the developer do not believe in the feasibility of ZooRes, because they think it is not possible to motivate 85-95% of the population to wear the TraT. Therefore, any secretary of a department may reject it.

The HHS secretary must have and know his or her modus operandi. His or her modus operandi may enable them to convince the public, but also discourage. The negative aspects of the modus operandi mustn't become the subject of the campaign.

Further, the campaign may be associated with norming. The campaign team educates the public on dealing with the TraT, supportive service during the quarantine time, and ZooReS privacy. The campaign invites everyone to share their concerns about the ZooReS Concept. This concern is addressed by the campaign team with respect.

Conspiracy theorists or pathogen deniers may reject the ZooReS concept. Converting this group is impossible. They should propose a solution. Their solution must ensure the rights of ZooReS proponents not to be infected. The opponents could propose to stay in quarantine until the elimination is successful. The campaign team must accept such proposals because it does not prevent the elimination. This example shows what the developer means by respectful

The campaign team points out to opponents and supporters of the ZooReS that they have a common goal.

The HHS Secretary points out the benefits of the ZooRes to the population. The benefits may include a safe workplace because there is no fear of company closures, a prospect of life before emergence of the disease, the time in quarantine is shortened by the latency time. You may move freely by the length of the latency time, his family member may be embellished from the illness because he or she receives the notice in time, avoiding infected persons in time so that no restriction of his freedom of movement due to quarantine, the likelihood of dying from an infectious disease is minimized, and need to take the risks of vaccines is not present.

The HHS Secretary points out the disadvantages of the IDDS to the individual. The advantages are the following:
Loss of freedom of movement for a few days for a small number of persons
Prevent the attendance of a unique or important event
prevents to exploit a business opportunity
Puts you in an uncomfortable situation
Need for a demilitarized zone to solve the cross-border problem Do the benefits outweigh the disadvantages? Yes, but it would be wrong to assume that everyone thinks the same. The modus operandi in this case is common sense is not common. The accomplishment of a Hercules task lies before the secretary of HHS.

The neighboring state does not have ZooReS in use. Cross-border commuters are indispensable for the economy. Consequently, border crossers undermine elimination efforts. The demilitarized zone allows border crossers to enter only this zone. The population living in the demilitarized zone may only enter the military zone after a quarantine period. Once again, the TraTs monitors compliance with the Demilitarized Zone. If a person from the militarized zone meets another person from the demilitarized zone then the status of the person from the militarized zone may change to quarantine.

For instance, if a person wants to live long and drinks alcohol excessively, eats excessively, and smokes, then the person lacks any common sense by this definition. If a person wants to live short and drinks alcohol excessively, eats and smokes, then the person has a lot of common sense by this definition.

The HHS manager's main task is to persuade the people to use this value-free definition as a guide to judge the argument of the proponent and opponents.

The HHS-Secretary has to overcome the economic, social, and political adversities to get the population involved in the initative of eliminating the pathogen. The HHS Secretary does not need the extreme left or right to achieve the goals of the campaign. He cannot convince these groups to adopt the definition of common sense. He does, however, need the support of the left, the apolitical, and the right. In other words, the Democrats, nonpartisan and Republican. What is the modus operandi of the democrats and republicans?

Thus, Republicans favor shifting money from welfare to warfare, because they think national defense is a vital responsibility of the government whereas the welfare state stifles market forces and individual freedom. (Loren B. Thompson, 2008) (8) Democrats, on the other hand, usually want to shift funding from warfare to welfare, because they favor a more expansive role for the federal government in supplementing and regulating the private economy. (Loren B. Thompson, 2008)(8)

These words warfare, private economy, and funding help identify the major allies of the HHS-Secretary such as:
Who is in charge of warfare? Secretary of Defense
Who is in charge of welfare? Secretary of Health and Human Services
Who is in charge of the private economy? Secretary of commerce
Who is in charge of funding? Secretary of treasury

This is the campaign team that is going to make the impossible possible. This team ignites the sting of battle in Americans to start rolling against the pathogen. The key to the success of the ZooReS is a staff with expertise in disease control, a campaign team committed to disease control, effective legal support from the District Attorney's office, and the political support of the population.

The gonorrhea control program described by Potterat et al. is a valuable model of the use of public health enforcement powers and it also is a powerful cautionary tale for the future of effective public health enforcement in the United States. The key to the success of this program was a staff with expertise in disease control, a health director committed to disease control, effective legal support from the District Attorney's office, and the political support of the community. (Richards & Rathbun, 1999)(21)

The American Civil War represents a landmark in military and medical history as the last large-scale conflict fought without knowledge of the germ theory of disease. Unsound hygiene, dietary deficiencies, and battle wounds set the stage for epidemic infection, while inadequate information about disease causation greatly hampered disease prevention, diagnosis, and treatment. Pneumonia, typhoid, diarrhea/dysentery, and malaria were the predominant illnesses. Altogether, two-thirds of the approximately 660,000 deaths of soldiers were caused by uncontrolled infectious diseases, and epidemics played a major role in halting several major campaigns. These delays, coming at a crucial point early in the war, prolonged the fighting by as much as 2 years. (Sartin, 1993)(35)

What is the combat capability of the U.S. armed forces when the U.S. armed forces are only half manned due to an epidemic? Worse still is the morale of the U.S. armed forces when the men and women know they cannot receive medical care due to war injuries because the hospitals at home have no capabilities If a war is underway and an epidemic breakout and the enemy has a ZooReS then the war becomes an indirect biological war. Indirect because the enemy did not employ it. may the US win a biological war without a biological defense system like the ZooReS? This question must be the subject of the discussion with the opponents. The task for the Secretary of Defense is to explain to the people that this is unlikely to succeed. Therefore, everyone must show the willingness to carry the TraT.

Funding for the elimination campaign may come from the Department of Defense. This may please the Democrats. The population should know that the elimination of diseases is a national security issue.

Being a national security issue should trigger the fear reflex of the Chinese and Russians. The Americans are building a biological defense system and the next step may be biological weapons. This biological weapon could be used by the Americans against us. Therefore, we must also build ZooReS. This may be the start of a worldwide disease eradication system. The exemplary role of the source of US funding signals the poor countries to fund their ZooReS from their defense budget. A similar percentage reduction of the military budget of all nations leads to the preservation of the existing military balance. The problem of rich and poor does not exist.

The secretary of defense makes or breaks the sting of battle going in the Americans. The model of funding the ZooReS may seem awkward but the developer relies on the knowledge of President Eisenhower.

Every gun that is made, every warship launched, every rocket fired signifies in the final sense, a theft from those who hunger and are not fed, those who are cold and are not clothed. This world in arms is not spending money alone. It is spending the sweat of its laborers, the genius of its scientists, the hopes of its children. This is not a way of life at all in any true sense. Under the clouds of war, it is humanity hanging on a cross of iron.” (Eisenhower)

Strategic goals of the US. Department of Commerce may include one overarching goal: Helping the American Economy Grow. Each of the Department's five strategic goals advances our mission and supports this goal. Further, the five strategic goals may include accelerating American leadership, enhancing job creation, strengthening US economic and national security, fulfilling constitutional requirements and support economic activity, and delivering customer-centric service excellence (Strategic goals)

A pandemic prevents the achievement of the five strategic goals because if the jobs are not manned the economy does not grow and opportunities fail to occur. The Secretary of Commerce role is to ensure the support of the ZooReS through the trade associations.

Ideally, CEOs could suggest private procurement of such a system. Private procurement does not lead to elimination. Therefore, government procurement is to be preferred. Entrepreneurs show their staff the advantages of the ZooReS, especially the protection of the workplace.

Theoretically, the Secretary of Commerce could solve the problem with the Chambers of Commerce. The problem is 90% should carry the Trat when they meet someone who does not live in the household. Each business leader only has to convince his cadres to carry the TraT. His cadres only have to convince their employees to wear the TraT and the employee has to convince all his family members to wear the TraT. The current unemployment rate in the US is about 11 percent. Presumably, this method would reach 89% of the citizens, and by their consent, 89% of the population would wear the device. This idea could lead some to link employment to the wearing of the device. The developer advises against this because any logic may be fooled.

The current secretary of commerce Gina Raimondo has this sting of battle or necessary modus operandi for this campaign. Quotes of the current secretary of commerce (13):
Because when we come together as a country there is nothing that may stop us.
We may meet these unprecedented challenges
As the secretary of commerce, I am committed to helping working Americans and businesses—small and large—to combat this pandemic head-on. (Secretary Raimondo)

The federal government has spent $3.00 trillion in response to COVID-19. If it goes on forever, the number may reach a level that may lead to an over-indebtedness of the state. The role of the Secretary of Finance is to explain the critical situation to the population. Bring representatives of American Families, Works, Small Businesses, State, Local, Tribal Governments, and American Industry in front of the camera and let them tell their stories. Above all, let them tell what it would have meant if there had been no support.

The secretary of HHS does not sugarcoat what quarantine and disruption of plans may be like for the population.

Social consequences of mass quarantine: We identified seven types of social consequences of mass quarantine (Table 4): psychological distress (n=11), heightened communication inequalities (n=9), food insecurity (n=8), economic challenges (n=7), diminished access to health care (n=6), disruptive education (n=4) and gender inequity and violence (n=3) (Isaac Yen-Hao Chu, 2020). (12)

| Consequences | | | Recommendations |
|---|---|---|---|
| identified from 15 | | | from 15 included |
| included studies | Themes | Examples | studies |
| Psychological | Emotional | Annoyance, anxiety, | Provide both |
| and mental | conditions | boredom, | personal |
| distress | | disappointment, fear | consultations and |
| | | of infection, | community |
| | | isolation, loneliness, | psychological |
| | | and mistrust | support to |
| | | | vulnerable |
| | | | populations |
| | Symptoms of | Post-traumatic | |
| | mental | stress disorder and | |
| | disorders | depressive | |
| | | disorders | |
| | Stigma and | Self-isolated | |
| | discrimination | individuals and | |
| | | EVD survivors | |
| | | were regarded as | |
| | | EVD spreaders; | |
| | | anti-Asian racism | |
| | | during the SARS | |
| | | outbreak | |
| | | in Canada | |
| Heightened | Public distrust of | Growing distrust of | Provide |
| communication | governments' | governments' | comprehensive |
| inequalities | responses | compulsory | support and |
| | | lockdown of slums in | transparent |
| | | Liberia's EVD | information on |
| | | outbreak | quarantine |
| | | | Combating |
| | | | misinformation by |
| | | | adopting context- |
| | | | specific approaches |
| | | | and supporting |
| | | | research efforts |
| | | | Prevent |
| | | | implementation |
| | | | failure by engaging |
| | | | with socially |
| | | | vulnerable |
| | | | populations |
| | Misinformation | Contradictory | |
| | on quarantine | quarantine | |
| | measures | instructions from | |
| | | public health | |
| | | officials, mass media | |
| | | and unauthorized | |
| | | analysts during | |
| | | Canada's SARS | |
| | | outbreak | |
| | Limited compliance | Overcrowding, | |
| | to quarantine orders | poverty and lack of | |
| | with increased risks | health care were | |
| | of health | reported as | |
| | | determinants of | |
| | | individuals' | |
| | | compliance to | |
| | | quarantine in EVD, | |
| | | H1N1 and SARS | |
| | | outbreaks | |
| Food | Food production | Little grain | Nutritional |
| insecurity | and transportation | harvesting during | preparedness, such |
| | | EVD confinement in | as food production, |
| | | Sierra Leone; | access, distribution |
| | | delayed food | and monitoring |
| | | transportation due | should be planned |
| | | to travel restrictions | and timely |
| | | for SARS | implemented |
| | | containment in | |
| | | China | |
| | Food access and | Reduced access to | |
| | storage | food during mass | |
| | | quarantine against | |
| | | SARS in China and | |
| | | Canada as well as | |
| | | EVD in Sierra Leone | |
| | | and Liberia | |
| Economic | Interrupted | Agricultural | Provide equitable |
| challenges | international | production, leisure | financial |
| | industries | business and tourism | compensation (e.g. |
| | | at domestic and | universal credits or |
| | | international levels | extensions of |
| | | during EVD and | business relief) to |
| | | SARS outbreaks | ensure the financial |
| | | | security of those |
| | | | under quarantine |
| | Closure of local | Shutdown of non- | |
| | business entities | essential business | |
| | | and reduced | |
| | | business revenue | |
| | | due to decreased | |
| | | demands with | |
| | | existing costs of | |
| | | employment in | |
| | | H1N1 preparedness | |
| | | plans | |
| | Reduced personal | Unemployment and | |
| | incomes | unstable incomes for | |
| | | part-time or are self- | |
| | | employed | |
| | | individuals during | |
| | | Canada's SARS | |
| | | outbreak | |
| Diminished | Access to essential | Increased number of | Strengthen the |
| access to | medicine and | deaths and | capacity of health |
| health care | services for non- | complications from | care systems and |
| | communicable | preventable health | equitable health care |
| | diseases | conditions during | access |
| | | EVD outbreak in | |
| | | Liberia; lacked | |
| | | access to regular | |
| | | prescriptions in | |
| | | Canada's SARS | |
| | | outbreak and | |
| | | China's COVID-19 | |
| | | outbreak | |
| | Reduced health- | Drop-outs of | |
| | seeking behavior | nutrition screening | |
| | | and hiding treatable | |
| | | illnesses during | |
| | | EVD outbreaks in | |
| | | Sierra Leone and | |
| | | Liberia | |
| Disruptive of | Remote and online | Web-based learning | Ensure resource |
| education | education | resources for | allocation for |
| | | adolescents and | education |
| | | students during | innovation |
| | | SARS outbreak in | and platforms |
| | | Canada | |
| | Caregivers as | Community members | |
| | educators | took responsibility | |
| | | for children's | |
| | | education during | |
| | | Liberia's EVD | |
| | | outbreak and | |
| | | Canada's H1N1 | |
| | | outbreak | |
| Gender | Gendered home care | Women's default | |
| inequity and | responsibility | role as caregivers at | |
| violence | | home during the | |
| | | EVD outbreak in | |
| | | Liberia | |
| | Gender-based | Increased numbers | Establish gender- |
| | violence | of women | inclusive norms in |
| | | experiencing | national |
| | | domestic violence in | policymaking and |
| | | China and the UK | global health |
| | | during the COVID- | governance |
| | | 19 pandemic | |

The Secretary of Health establishes services to address the negative effects of mass quarantine. The suspect calls and describes his problem to a member of the service. It is the task of the employee to propose a solution. This solution must be feasible for the suspect. For example, an entrepreneur calls the service. An employee is contagious and 10 of his colleagues are suspicious. These 10 colleagues must complete the order in time else the entrepreneur must pay a 1-million-dollar conventional penalty per day of delay. The two days quarantine leads to a two days delay. Furthermore, his reputation is down the drain. It becomes a threat to the existence of the company. It is up to the employee of the service to propose and implement the following idea. During the two days quarantine, the 10 persons work together but stay in a hotel isolated from the public. The costs of accommodation and transport may be covered by the health authority.

Success demands a high level of logistical and organizational competence. (Patton)

There may be numerous unpredictable scenarios to solve. Setting up an organization to handle this scenario may task the logistical and organizational competence of the Secretary of Health.

People believe what they want. It is a waste of time, money, and energy to try to change these beliefs. The Secretary of Health needs the help of the Physicians and Nurses Association, Universities, and Civil Rights Organizations. Ideally, the physicians and nurses' association may manage the elimination of the pathogen and the Department of Defense may pay for this service. The physician association would be responsible for pathogen elimination and the nurse association run the services. The universities and civil rights organizations would be responsible for adhering to privacy regulations. There may be computer scientists who believe a system for contact tracing without personal data and central data storage is impossible. The university may offer lectures to computer scientists to explain the concepts.

Further, in an instance, a person wants to text a friend that I am running late for dinner. Further, the person may type up the message, “Hello! Sorry, I'm running late. Be there in 10.”

First, the phone converts your message into the GSM 7-bit alphabet.

Without going into too much detail, this alphabet takes the words in the above message and turns them into the tiniest bits of data that may then be transmitted to a cell tower.

This includes the message and some basic info about it such as its timestamp and the recipient's phone number.

Once the cell tower receives the message, it sends it to a system called the Short Message Service Center (SMSC).

The SMSC checks who the friend—the recipient—is and then checks if they are in range. If they are, the message is then sent to the tower nearest to them and then, finally, their mobile phone.

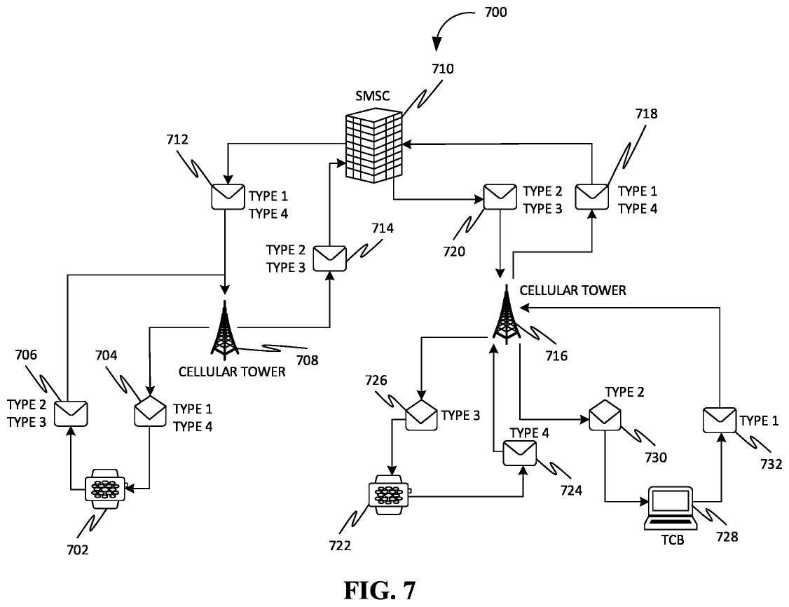

Further, the friend's phone then turns the messages back from the GSM 7-bit alphabet into characters by using the same code, but in reverse. What's particularly neat is that if the SMSC can't find the phone, the message may just bounce around in its system until the moment the phone reconnects, at which point it sends it immediately. (Wilkinson) (23). Further, as shown in FIG. 7, devices TraT and TCB use SMS to communicate. The following types of communication exist:

Type 1: TCB requests the TraT for service.

Type 2: TraT communicates to the TCB the result of the requested service.

Type 3: TraT requests another TraT for a service involving TCB.

Type 4: TraT sends the result of the service to TraT involving TCB.

The TraT may be switched on to communicate with TCB only 1 time per day for 5 to 10 minutes. Thus, the TraT saves power and the persons are exposed to the electromagnetic field only for a short time. The SMSC stores the message until the TraT is on.

The TraT-ID is a cell phone number. If one TraT knows the TraT-ID of another, they may communicate. A TCB may communicate a request to a TraT if the TCB knows the TraT-ID of the TraT.

When a TCB makes a request, the phone number of the TCB is always communicated to the TraT.

Further, the TraT records all encounters with other TraT. Each TraT transmits radio waves with the defined social distancing range. This radio wave contains a phone number (TraT-ID). Communication with this TraT is possible only through this phone number. If several TraTs meet within a radius of the defined range, then they all dip out their phone number to each other. The recording is done until the defined range is exceeded. The TraTs stores the phone number, signal strength, and the time of receipt. The TraT also exchanges their separation value. One TraT warns the carrier of another TraT with the status True or false. The TraT of the uncontaminated person may change its separation to True if the uncontaminated ignores the warning for a defined time.

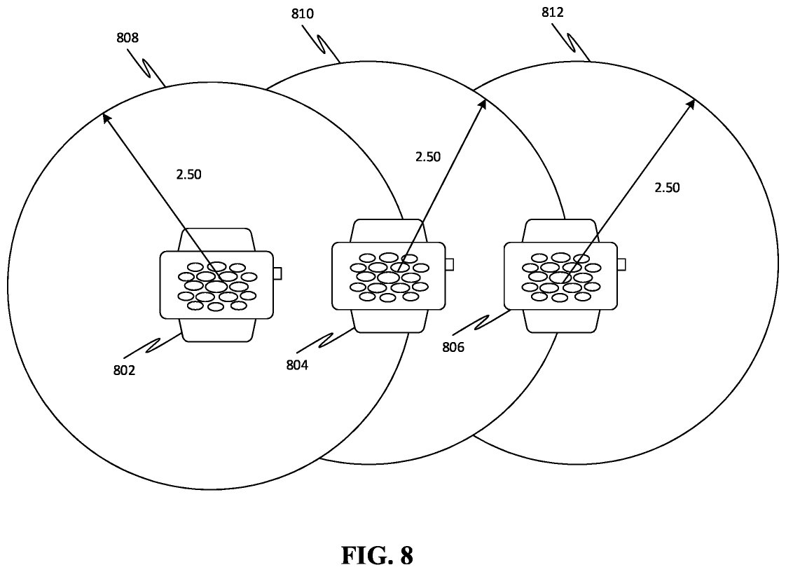

Further, as shown in FIG. 8, the left blue, and middle green TraT exchange their phone number and statuses. There is no phone number exchange between left blue and right red TraT. The green and red TraT exchange their phone number. The fact that objects and crowds reduce the signal strength mirrors the actual conditions of the pathogen. The pathogen also cannot pass through objects and its range of spreading is reduce by the contaminated person surrounded by people. The problem with carrying devices in the pocket or bag does not exist because the device is worn visibly. Telephone transmissions longer than 2 minutes are recorded. Therefore, passers-by are not registered.

Unlike common contact tracers, the distance between two TraT is not decisive for registration (Biddle, 2020)(25). TraTs decides whether to register another TraT if the distance between the two TraTs may be bridged by the pathogen. Therefore, if the radio wave cannot bridge the distance, then neither may the pathogen. This is more consistent with reality than the distance measurement.

False positives are possible. Among other things, this belongs to the collateral damage. When wearing the TraT correctly, a false negative is unthinkable. This circumstance of wrong wearing is minimized by possible social control. The device indicates whether someone is wearing a device or not. This is limited to two persons.

A TraT has a transmitter and a receiver. When TraTs transmit and receive there is a risk of collision. A collision is when several TraTs send their TraT-ID and the TraT-iD arrives at the receiver at the same time. Transmitting and receiving require a lot of power. It shortens the life of the battery. Therefore, it is not useful to work with the same transmitting and receiving power when no one is around.

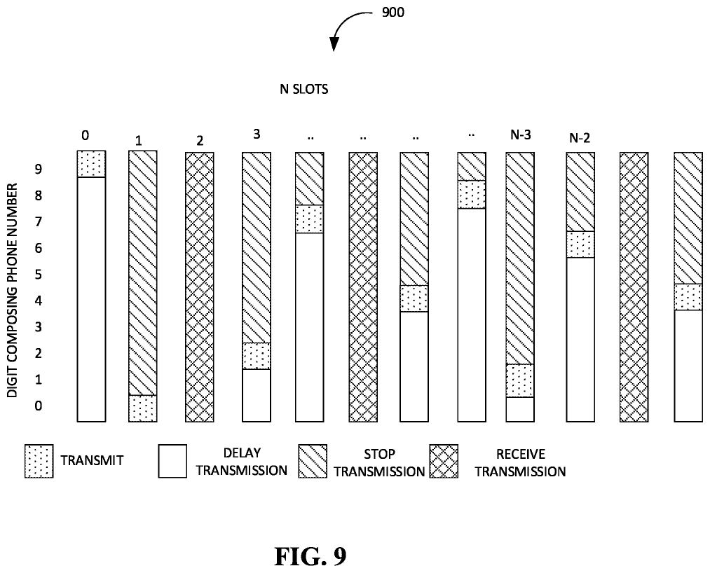

Further, as shown in FIG. 9, Each digit of the TraT-ID gets a time window and m number of receiver slots. A phase consists of the TraT-ID time window and receiver time slot.

The length of the time window is ten times the length to transmit the TraT-ID once. The length of a green box. Each time slot contains a time to send. The time is determined by the digit. In the diagram, the telephone number is 902737165. In the diagram, this point of time is marked with a green box. Before the TraT-ID may be sent, the transmitter must wait.

Drawn in the diagram with a red box. After sending, the sender waits until others have also sent messages. In the diagram drawn with the color yellow. The receiver time slots are randomly scattered in a phase. The receiver is always locked in during a receiver time window.

When no one is around, the transmitter may turn on once or twice during a phase. As soon as a person is near, the number of transmissions increases in one phase. As soon as no one is around, the number of transmissions in the phase decreases.

As described by the ITU, the E.164 general format must contain only digits split as follows: country code (1 to 3 digits) and subscriber number (max 12 digits). An encounter longer than 2 to 3 minutes is to be recorded. In the case of the international telephone number, the number of digits is given. As the only free variable, the number of time windows for receiving is to be determined experimentally.

Other primary keys like mac-address may be used but must be mapped to a telephone number. The TraTs would exchange the MAC addresses and ask TCB for the resolution.

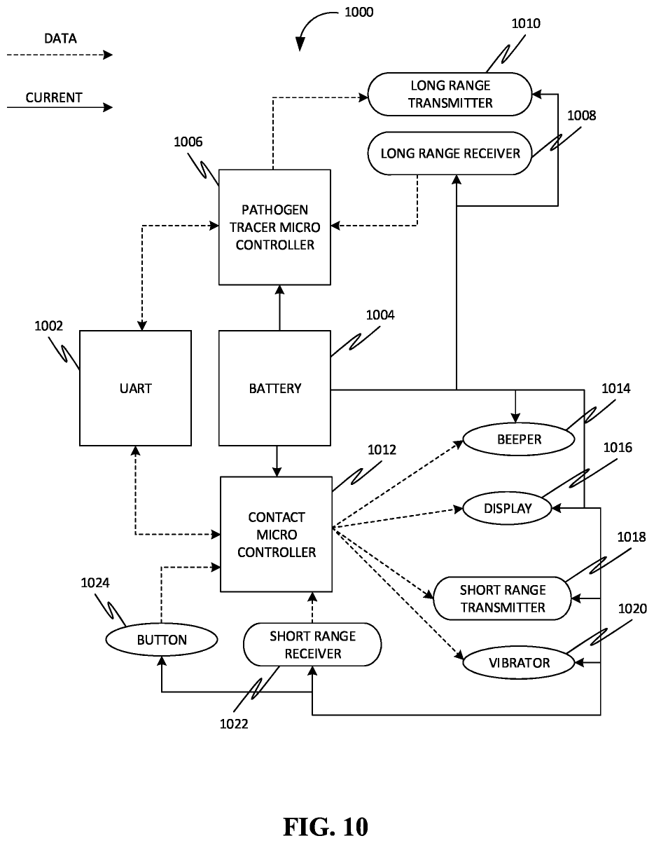

Further, as shown in FIG. 10, the pathogen tracer microcontroller executes the algorithm. The contract microcontroller executes an algorithm associated with the disclosed system. Two microcontrollers are used to save power. The pathogen microcontroller with its part system is normally only in use a few minutes a day. Therefore, it is in sleep mode. The parallel task order ensures that the recording of contact is not prevented by computationally intensive

Further, a vibrator associated with the disclosed device vibrates if the TraT receives the status True from a TraT of contact or contaminated person. The vibration continues until the wearer has reached a spot where the TraT does not receive the broadcast.

The wearer (or user) should check at meeting the operational status of the TraT. Green LED light indicates the team is registered. Red LED light indicates the status is True.

Further, a Status Button associated with the disclosed device may reset the TraT if pressed twice within 1 second. The red light is turn off once it is on and the Button is pressed. If the TraT vibrates press the button to stop the vibration.

Humanity is not hopelessly exposed to the dangers of zoonosis diseases. The elimination strategy is the most promising approach. The zero strategy countries like Australia, Sud Korea, and New Zealand have not achieved the breakthrough because they do not have ZooReS in use. The mitigating strategy Countries need to rethink their strategy. The success of the elimination strategy depends on the people's may to fight and the sincerity of the politicians. The technical, logistical, and organizational challenges are no more significant than those faced by armies. Therefore, the solutions to these challenges are known.

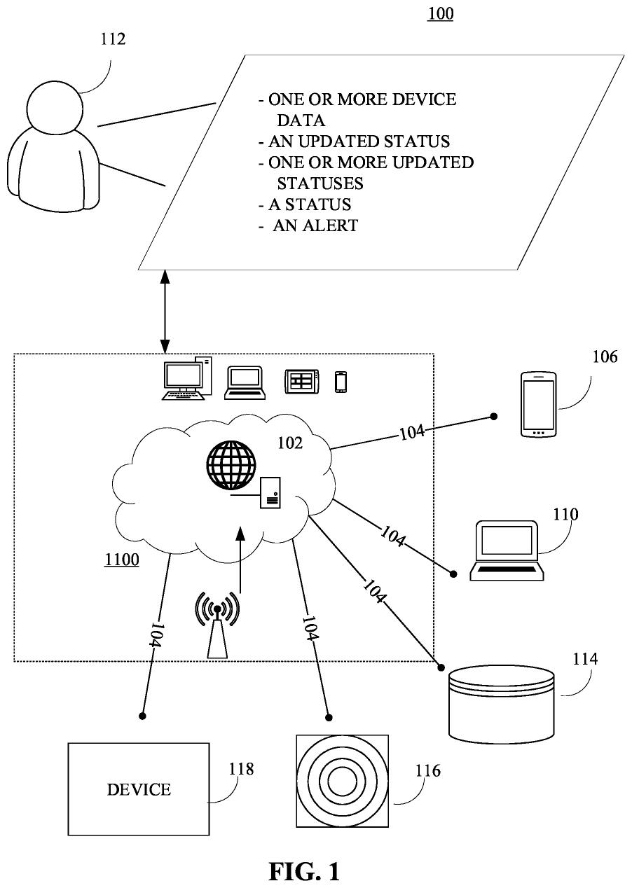

FIG. 1 is an illustration of an online platform 100 consistent with various embodiments of the present disclosure. By way of non-limiting example, the online platform 100 to facilitate managing a spread of infectious diseases among users may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers, etc.), databases 114, sensors 116, and a device 118 (such as a device 200 for managing a spread of infectious diseases among users) over a communication network 104, such as, but not limited to, the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

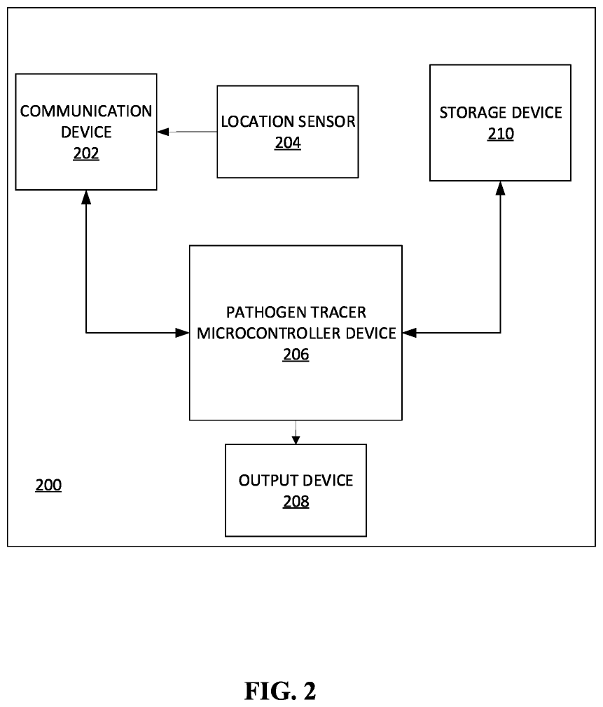

A user 112, such as the one or more relevant parties, may access online platform 100 through a web based software application or browser. The web based software application may be embodied as, for example, but not be limited to, a website, a web application, a desktop application, and a mobile application compatible with a computing device 1100. FIG. 2 is a block diagram of a device 200 for managing a spread of infectious diseases among users, in accordance with some embodiments. Further, the device 200 may be associated with a user. Further, the device 200 may include a communication device 202, a location sensor 204, a pathogen tracer microcontroller device 206, an output device 208, and a storage device 210. Further, the communication device 202 may be configured for receiving one or more device data from one or more devices associated with one or more users. Further, the one or more device data may include one or more location data associated with one or more locations of the one or more devices corresponding to one or more time periods. Further, the location sensor 204 may be configured for generating location data based on a location of the device 200 corresponding to a time period. Further, the pathogen tracer microcontroller device 206 may be communicatively coupled with the communication device 202. Further, the pathogen tracer microcontroller device 206 may be configured for analyzing the location data and the one or more location data. Further, the pathogen tracer microcontroller device 206 may be configured for determining a spatiotemporal proximity of the device 200 to the one or more devices based on the analyzing. Further, the spatiotemporal proximity may include a proximity in distance and time. Further, the spatiotemporal proximity may include a nearness in distance between the user to the one or more users and a length of time period corresponding to the nearness. Further, the pathogen tracer microcontroller device 206 may be configured for comparing the spatiotemporal proximity with a predetermined spatiotemporal proximity based on the determining of the spatiotemporal proximity. Further, the predetermined spatiotemporal proximity may include a proximity in distance and time to be maintained between the user and the one or more users to prevent the spread of the infectious disease. Further, the predetermined spatiotemporal proximity may include a nearness in distance between the user and the one or more users and a length of time period corresponding to the nearness. Further, the pathogen tracer microcontroller device 206 may be configured for determining a risk of the spread of a pathogen causing an infectious disease between the user and the one or more users based on the comparing. Further, the pathogen tracer microcontroller device 206 may be configured for generating an alert based on the determining of the risk. Further, the output device 208 may be communicatively coupled with the pathogen tracer microcontroller device 206. Further, the output device 208 may be configured for producing one or more alert signals based on the alert. Further, the storage device 210 may be communicatively coupled with the pathogen tracer microcontroller device 206. Further, the storage device 210 may be configured for storing the alert. Further, the storage device 210 may be configured for storing a status of the user corresponding to the infectious disease. Further, the status may include a infected status and a not infected status.

Further, in some embodiments, the pathogen tracer microcontroller device 206 may be configured for identifying one or more first users from the one or more users based on the comparing. Further, the infectious disease may be likely to spread between the user and the one or more first users. Further, the generating of the alert may be based on the identifying.

Further, in some embodiments, the one or more device data may include one or more statuses of the one or more users. Further, the pathogen tracer microcontroller device 206 may be configured for identifying one or more first statuses of the one or more first users from the one or more statuses based on the identifying of the one or more first users. Further, the output device 208 may be configured for displaying the one or more first statuses of the one or more first users.

Further, in some embodiments, the storage device 210 may be configured for retrieving the status of the user based on the determining of the spatiotemporal proximity. Further, the pathogen tracer microcontroller device 206 may be configured for analyzing the status and the one or more statuses. Further, the determining of the risk may be based on the analyzing of the status and the one or more statuses.

Further, in some embodiments, the pathogen tracer microcontroller device 206 may be configured for updating at least one of the status and the one or more statuses based on the determining of the risk. Further, the pathogen tracer microcontroller device 206 may be configured for generating at least one of an updated status of the user corresponding to the infectious disease and one or more updated statuses of the one or more users corresponding to the infectious disease based on the updating. Further, the storage device 210 may be configured for storing at least one of the updated status and the one or more updated statuses.

Further, in some embodiments, the communication device 202 may be configured for transmitting at least one of the updated status and the one or more updated statuses to the one or more devices.

Further, in some embodiments, the storage device 210 may be configured for retrieving the status of the user based on the determining of the spatiotemporal proximity. Further, the communication device 202 may be configured for transmitting the status to the one or more devices.

Further, in some embodiments, the communication device 202 may be configured for transmitting the alert to the one or more devices. Further, the one or more devices may include one or more output devices. Further, the one or more output devices produces the one or more alert signals based on the alert.

Further, in some embodiments, the one or more device data may include one or more device identifiers of the one or more devices, one or more spatiotemporal identifiers of the one or more devices, and one or more statuses of the one or more users associated with the one or more devices. Further, the pathogen tracer microcontroller device 206 may be configured for analyzing the one or more device identifiers, the one or more spatiotemporal identifiers, and the one or more statuses. Further, the pathogen tracer microcontroller device 206 may be configured for identifying one or more contaminated users from the one or more users based on the analyzing of the one or more device identifiers, the one or more spatiotemporal identifiers, and the one or more statuses. Further, the pathogen tracer microcontroller device 206 may be configured for generating a first alert for the one or more contaminated users based on the identifying of the one or more suspected users. Further, the output device 208 may be configured for producing one or more first alert signals based on the first alert.

Further, in some embodiments, the pathogen tracer microcontroller device 206 may be configured for generating one or more recommendations to prevent the risk of the spread of the pathogen causing the infectious disease based on the determining of the risk. Further, the output device 208 may be configured for displaying the one or more recommendations to the user.

In further embodiments, the device 200 may include a wearable device. Further, the wearable device may be configured to be worn on at least a part of a body of the user.

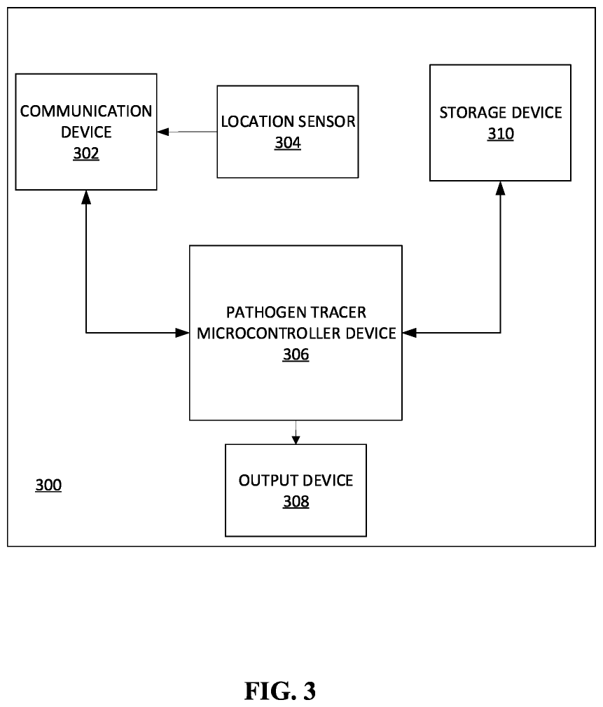

FIG. 3 is a block diagram of a device 300 for managing a spread of infectious diseases among users, in accordance with some embodiments. Further, the device 300 may be associated with a user. Further, the device 300 may include a communication device 302, a location sensor 304, a pathogen tracer microcontroller device 306, an output device 308, and a storage device 310.

Further, the communication device 302 may be configured for receiving one or more device data from one or more devices associated with one or more users. Further, the one or more device data may include one or more location data associated with one or more locations of the one or more devices corresponding to one or more time periods. Further, the location sensor 304 may be configured for generating location data based on a location of the device 300 corresponding to a time period.

Further, the pathogen tracer microcontroller device 306 communicatively coupled with the communication device 302. Further, the pathogen tracer microcontroller device 306 may be configured for analyzing the location data and the one or more location data. Further, the pathogen tracer microcontroller device 306 may be configured for determining a spatiotemporal proximity of the device 300 to the one or more devices based on the analyzing. Further, the pathogen tracer microcontroller device 306 may be configured for comparing the spatiotemporal proximity with a predetermined spatiotemporal proximity based on the determining of the spatiotemporal proximity. Further, the pathogen tracer microcontroller device 306 may be configured for determining a risk of the spread of a pathogen causing an infectious disease between the user and the one or more users based on the comparing. Further, the pathogen tracer microcontroller device 306 may be configured for identifying one or more first users from the one or more users based on the comparing. Further, the infectious disease may be likely to spread between the user and the one or more first users. Further, the pathogen tracer microcontroller device 306 may be configured for generating an alert based on the determining of the risk and the identifying.

Further, the output device 308 may be communicatively coupled with the pathogen tracer microcontroller device 306. Further, the output device 308 may be configured for producing one or more alert signals based on the alert.

Further, the storage device 310 may be communicatively coupled with the pathogen tracer microcontroller device 306. Further, the storage device 310 may be configured for storing the alert. Further, the storage device 310 may be configured for storing a status of the user corresponding to the infectious disease.

Further, in some embodiments, the one or more device data may include one or more statuses of the one or more users. Further, the pathogen tracer microcontroller device 306 may be configured for identifying one or more first statuses of the one or more first users from the one or more statuses based on the identifying of the one or more first users. Further, the output device 308 may be configured for displaying the one or more first statuses of the one or more first users.

Further, in some embodiments, the storage device 310 may be configured for retrieving the status of the user based on the determining of the spatiotemporal proximity. Further, the pathogen tracer microcontroller device 306 may be configured for analyzing the status and the one or more statuses. Further, the determining of the risk may be based on the analyzing of the status and the one or more statuses.

Further, in some embodiments, the pathogen tracer microcontroller device 306 may be configured for updating at least one of the status and the one or more statuses based on the determining of the risk. Further, the pathogen tracer microcontroller device 306 may be configured for generating at least one of an updated status of the user corresponding to the infectious disease and one or more updated statuses of the one or more users corresponding to the infectious disease based on the updating. Further, the storage device 310 may be configured for storing at least one of the updated status and the one or more updated statuses.

Further, in some embodiments, the communication device 302 may be configured for transmitting at least one of the updated status and the one or more updated statuses to the one or more devices.

Further, in some embodiments, the storage device 310 may be configured for retrieving the status of the user based on the determining of the spatiotemporal proximity.

Further, the communication device 302 may be configured for transmitting the status to the one or more devices.

Further, in some embodiments, the communication device 302 may be configured for transmitting the alert to the one or more devices. Further, the one or more devices may include one or more output devices. Further, the one or more output devices produces the one or more alert signals based on the alert.

Further, in some embodiments, the one or more device data may include one or more device identifiers of the one or more devices, one or more spatiotemporal identifiers of the one or more devices, and one or more statuses of the one or more users associated with the one or more devices. Further, the pathogen tracer microcontroller device 306 may be configured for analyzing the one or more device identifiers, the one or more spatiotemporal identifiers, and the one or more statuses. Further, the pathogen tracer microcontroller device 306 may be configured for identifying one or more contaminated users from the one or more users based on the analyzing of the one or more device identifiers, the one or more spatiotemporal identifiers, and the one or more statuses. Further, the pathogen tracer microcontroller device 306 may be configured for generating a first alert for the one or more contaminated users based on the identifying of the one or more suspected users. Further, the output device 308 may be configured for producing one or more first alert signals based on the first alert.

Further, in some embodiments, the pathogen tracer microcontroller device 306 may be configured for generating one or more recommendations to prevent the risk of the spread of the pathogen causing the infectious disease based on the determining of the risk. Further, the output device 308 may be configured for displaying the one or more recommendations to the user.

FIG. 4 is a block diagram of a system 400 for managing a spread of infectious diseases among users, in accordance with some embodiments. Accordingly, the system 400 may include a proximity sensor 402, human being sensor 404, an authentication sensor 406, a Bluetooth transmitter 408, a Bluetooth receiver 410, a telephone receiver 412, CPU 414, a flash memory 416, a ROM 418, a RAM 420, a telephone transmitter 422, a vibrator device 424, and a display device 426. Further, the proximity sensor 402 may be associated with a proximity driver 428. Further, the human being sensor 404 may be associated with a H-B driver 430. Further, the authentication sensor 406 may be associated with an authentication driver 432. Further, the Bluetooth transmitter 408 and the Bluetooth receiver 410 may be associated with a Bluetooth driver 434. Further, the telephone receiver 412 may be associated with a T-R driver 436. Further, the telephone transmitter 422 may be associated with a T-T driver 438. Further, the vibrator sensor 424 may be associated with a vibrator driver 440. Further, the display device 426 may be associated with a display driver 446 Further, the system 400 may be associated with an operating system 442 associated with a virus control application 444.

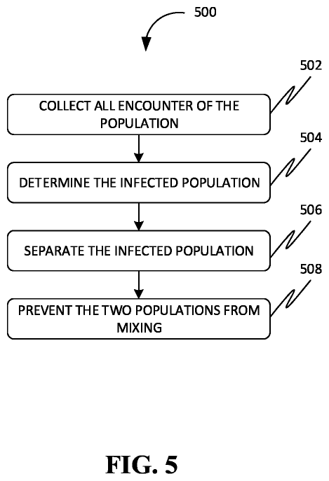

FIG. 5 is a flow chart of a method 500 for managing a spread of infectious diseases among users, in accordance with some embodiments. Further, at 502, the method 500 may include collecting all encounters of the population. Further, at 504, the method 500 may include determining the infected population. Further, at 506, the method 500 may include separating the infected population. Further, at 508, the method 500 may include preventing the two populations from mixing.

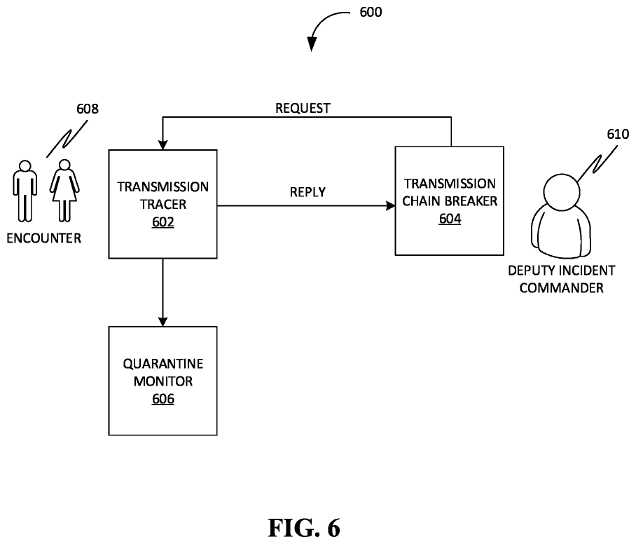

FIG. 6 is a block diagram of a system 600 for managing a spread of infectious diseases among users, in accordance with some embodiments. Further, the system 600 may include a transmission tracer (TraT) 602, a transmission chain breaker 604, and a quarantine monitor 606. Further, the transmission tracer 602 may trace an encounter 608 associated with an infected person and a non-infected person. Further, the transmission chain breaker 604 may be associated with a deputy incident commander 610.

FIG. 7 is a schematic of a system 700 for managing a spread of infectious diseases among users, in accordance with some embodiments. Further, the system 700 may include a first TraT device 702 communicatively coupled to a first cellular tower 708. Further, the first Trat device 702 may communicate a message 704 to the first cellular tower 708. Further, the message 704 may be type 1 and type 4. Further, the first Trat device 702 may communicate a message 706 to the first cellular tower 708. Further, the message 706 may be type 2 and type 3. Further, the first cellular tower 708 may be communicatively coupled to a Short Message Service Center (SMSC) 710. Further, the first cellular tower 708 may communicate a message 712 to the SMSC 710. Further, the message 712 may be type 1 and type 4. Further, the first cellular tower 708 may communicate a message 714 to the SMSC 710. Further, the message 714 may be type 2 and type 3. Further, the SMSC 710 may be communicatively coupled to a second cellular tower 716. Further, the SMSC 710 may communicate a message 718 to the second cellular tower 716. Further, the message 718 may be type 1 and type 4. Further, the SMSC 710 may communicate a message 720 to the second cellular tower 716. Further, the message 720 may be type 2 and type 3. Further, the system 700 may include a second TraT device 722 communicatively coupled to the second cellular tower 716. Further, the second cellular tower 716 may communicate a message 724 to the second TraT device 722. Further, the message 724 may be type 4. Further, the second cellular tower 716 may communicate a message 726 to the second TraT device 722. Further, the message 726 may be type 3. Further, the system 700 may include a TCB 728 communicatively coupled to the second cellular tower 716. Further, the second cellular tower 716 may communicate a message 730 to the TCB 728. Further, the message 730 may be type 2. Further, the second cellular tower 716 may communicate a message 732 to the TCB 728. Further, the message 732 may be type 1.

FIG. 8 illustrates a plurality of Trat devices 802-806 associated with the disclosed system, in accordance with some embodiments. Accordingly, a first TraT device 802 and a second TraT device 804 may exchange their phone number and statuses. There may be no phone number exchange between the first TraT device 802 and a third TraT device 806. The third TraT device 806 and the second TraT device 804 may exchange their phone number. Further, the first TraT device 802 may be associated with a diametrical range 808. Further, the second TraT device 804 may be associated with a diametrical range 810. Further, the third TraT device 806 may be associated with a diametrical range 812.

FIG. 9 is a graphical representation 900 illustrating a time window and number of receiver slots for a TraT device associated with the disclosed system, in accordance with some embodiments. Accordingly, the TraT device may be associated with a TraT-ID. Further, each digit of the TraT-ID gets a time window and m number of receiver slots. A phase consists of the TraT-ID time window and receiver time slot. Further, the length of the time window is ten times the length to transmit the TraT-ID once. Further, each time slot contains a time to send.

FIG. 10 is a schematic of a system 1000 for managing a spread of infectious diseases among users, in accordance with some embodiments. Accordingly, the system 1000 may include a UART 1002, a battery 1004, a pathogen tracer micro-controller 1006, a long-range receiver 1008, a long-range transmitter 1010, a contact micro-controller 1012, a beeper 1014, a display 1016, a short-range transmitter 1018, a vibrator 1020, a short-range receiver 1022, and a button 1024. Further, the battery 1004 may be electrically coupled to the pathogen tracer micro-controller 1006, the long-range receiver 1008, the long-range transmitter 1010, the contact micro-controller 1012, the beeper 1014, the display 1016, the short-range transmitter 1018, the vibrator 1020, the short-range receiver 1022, and the button 1024. Further, the pathogen tracer micro-controller 1006 may be communicatively coupled to the long-range receiver 1008, the long-range transmitter 1010, and the UART 1002. Further, the contact micro-controller 1012 may be communicatively coupled to the UART 1002, the beeper 1014, the display 1016, the short-range transmitter 1018, the vibrator 1020, the short-range receiver 1022, and the button 1024.

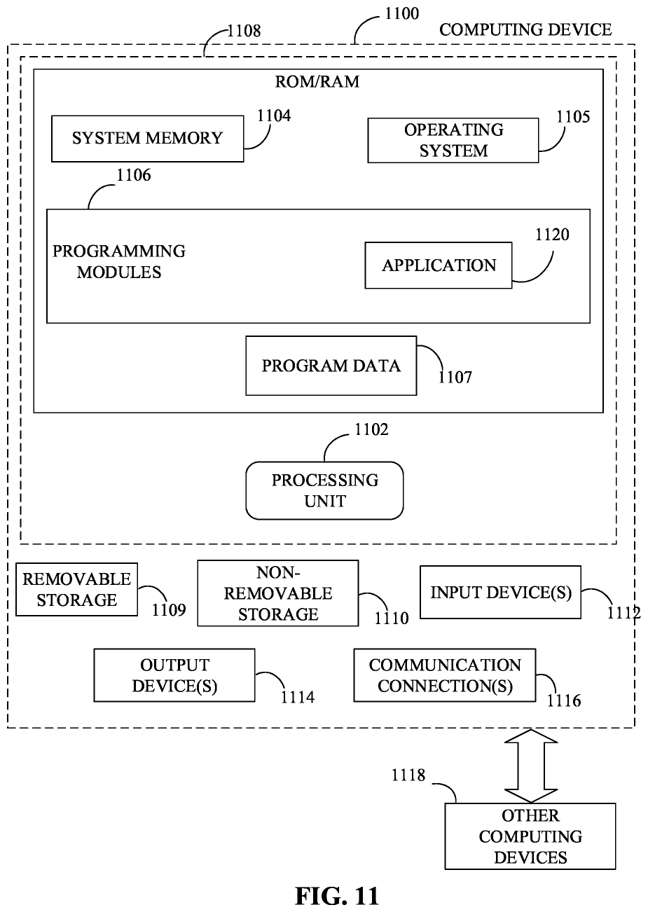

With reference to FIG. 11, a system consistent with an embodiment of the disclosure may include a computing device or cloud service, such as computing device 1100. In a basic configuration, computing device 1100 may include at least one processing unit 1102 and a system memory 1104. Depending on the configuration and type of computing device, system memory 1104 may comprise, but is not limited to, volatile (e.g. random-access memory (RAM)), non-volatile (e.g. read-only memory (ROM)), flash memory, or any combination. System memory 1104 may include operating system 1105, one or more programming modules 1106, and may include a program data 1107. Operating system 1105, for example, may be suitable for controlling computing device 1100's operation. In one embodiment, programming modules 1106 may include image-processing module, machine learning module. Furthermore, embodiments of the disclosure may be practiced in conjunction with a graphics library, other operating systems, or any other application program and is not limited to any particular application or system. This basic configuration is illustrated in FIG. 11 by those components within a dashed line 1108.

Computing device 1100 may have additional features or functionality. For example, computing device 1100 may also include additional data storage devices (removable and/or non-removable) such as, for example, magnetic disks, optical disks, or tape. Such additional storage is illustrated in FIG. 11 by a removable storage 1109 and a non-removable storage 1110. Computer storage media may include volatile and non-volatile, removable and non-removable media implemented in any method or technology for storage of information, such as computer-readable instructions, data structures, program modules, or other data. System memory 1104, removable storage 1109, and non-removable storage 1110 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which may be used to store information and which may be accessed by computing device 1100. Any such computer storage media may be part of device 1100. Computing device 1100 may also have input device(s) 1112 such as a keyboard, a mouse, a pen, a sound input device, a touch input device, a location sensor, a camera, a biometric sensor, etc. Output device(s) 1114 such as a display, speakers, a printer, etc. may also be included. The aforementioned devices are examples and others may be used.

Computing device 1100 may also contain a communication connection 1116 that may allow device 1100 to communicate with other computing devices 1118, such as over a network in a distributed computing environment, for example, an intranet or the Internet. Communication connection 1116 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1104, including operating system 1105. While executing on processing unit 1102, programming modules 1106 (e.g., application 1120) may perform processes including, for example, one or more stages of methods, algorithms, systems, applications, servers, databases as described above. The aforementioned process is an example, and processing unit 1102 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that may contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program may be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data may also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

Although the present disclosure has been explained in relation to its preferred embodiment, it is to be understood that many other possible modifications and variations may be made without departing from the spirit and scope of the disclosure.

### REFERENCES

1. Tejvan Pettinger. (2016, February 14). Economic uncertainty. Retrieved from economicshelp.org: https://www.economicshelp.org/blog/4941/economics/economic-uncertainty/
2. American Heroes. (n.d.). Retrieved from U.S. Department of the Interior: https://www.doi.gov/american-heroes/George-Patton
3. Ausmed Editorial Team. (2020, April 6). Infectious Diseases: How Do You Break the Chain? Retrieved from Ausmed: https://www.ausmed.com/cpd/articles/infectious-diseases
4. avira. (2020, June 15). report-majority-of-americans-wont-use-covid-contact-tracing-apps. Retrieved from https://www.avira.com/:https://www.avira.com/en/press/report-majority-of-americans-wont-use-covid-contact-tracing-apps
5. Basic Economic Problem. (n.d.). /basic-economic-problem. Retrieved from economicshelp.org: https://www.economicshelp.org/blog/glossary/basic-economic-problem/
6. bbc news. (2020, Mar. 16). Coronavirus: ‘We are at war’—Macron. Retrieved from bbc: https://www.bbc.com/news/av/51917380
7. Biddle, S. (2020, May 5). coronavirus-bluetooth-contact-tracing. Retrieved from theintercept.com: https://theintercept.com/2020/05/05/coronavirus-bluetooth-contact-tracing/
8. biography George Smith Patton. (n.d.). George-Smith-Patton/Controversies-and-appraisal. Retrieved from britannica.com:britannica.com/biography/George-Smith-Patton/Controversies-and-appraisal
9. Core Roles in Change Management. (n.d.). core-roles-in-change-management. Retrieved from Prosci: https://www.prosci.com/resources/articles/core-roles-in-change-management
10. Debt monetization. (n.d.). Debt_monetization#Indirect_forms_of_monetary_financing. Retrieved from en.wikipedia.org: https://en.wikipedia.org/wiki/Debt monetization#Forms_of_monetary_financing
11. diseases eliminated from the United States. (2015, February). List_of_diseases_eliminated_from_the_United_States. Retrieved from wikipedia.org/: https://en.wikipedia.org/wiki/List_of_diseases_eliminated_from_the_United_States
12. Eisenhower, D. D. (n.d.). dwight_d_eisenhower_149102. Retrieved from brainyquote.com: https://www.brainyquote.com/quotes/dwight_d_eisenhower_149102
13. Emergency Management and the Incident Command System. (n.d.). emergencymanagement. Retrieved from phe.gov: https://www.phe.gov/Preparedness/planning/mscc/handbook/chapter1/Pages/emergencymanagement.aspx
14. Isaac Yen-Hao Chu, M. P. (2020, October 13). 27/7/taaa192/5922349. Retrieved from academic.oup.com:https://academic.oup.com/jtm/article/27/7/taaa192/5922349
15. Joint statement by ILO, FAO, IFAD and WHO. (2020, Oct. 13). 13-10-2020-impact-of-covid-19-on-people's-livelihoods-their-health-and-our-food-systems. Retrieved from who.int:https://www.who.int/news/item/13-10-2020-impact-of-covid-19-on-people's-livelihoods-their-health-and-our-food-systems
16. Lewis, D. (2020, Dec. 17). magazine-assets. Retrieved from https://media.nature.com/:https://media.nature.com/original/magazine-assets/d41586-020-03518-4/d41586-020-03518-4.pdf
17. Loren B. Thompson, P. (2008, Feb. 25). the-role-of-party-politics-in-shaping-defense-prioritie. Retrieved from https://www.lexingtoninstitute.org/:https://www.lexingtoninstitute.org/the-role-of-party-politics-in-shaping-defense-priorities/
18. Marques, C. P. (2021, Apr. 3). The Zero Covid strategy protects people and economies more effectively. Retrieved from nstitutmolinari.org/:https://www.institutmolinari.org/2021/04/03/the-zero-covid-strategy-protects-people-and-economies-more-effectively/
19. Max Roser, S. O. (2018, October). Eradication of Diseases. Retrieved from Our WorldO in Data: https://ourworldindata.org/eradication-of-diseases#key-requirements-for-disease-eradication
20. McMahon, M. (2020, May 29). Why is uncertainty so damaging for the economy? Retrieved from economics observatory: https://www.economicsobservatory.com/why-uncertainty-so-damaging-economy
21. Megan Lowry, S. (2021, Jul. 1). fighting-vaccine-hesitancy-what-can-we-learn-from-social-science. Retrieved from nationalacademies.org: https://www.nationalacademics.org/news/2021/07/fighting-vaccine-hesitancy-what-can-we-learn-from-social-science
22. Modes of transmission. (n.d.). view.php?id=84&section=20.4.4. Retrieved from www.open.edu:https://www.open.edu/openlearncreate/mod/oucontent/view.php?id=84&section=20.4.4
23. Pandemic Intervals Framework. (n.d.). national-strategy. Retrieved from cdc.go: https://www.cdc.gov/flu/pandemic-resources/national-strategy/intervals-framework.html
24. Patton, G. (n.d.). George S. Patton Quotes. Retrieved from azquotes.com: https://www.azquotes.com/quote/834836
25. period, C. o. (2020, Mar. 4). File:Concept_of_incubation_period.svg. Retrieved from wikimedia.org:https://commons.wikimedia.org/wiki/File:Concept_of incubation_period.svg
26. Pettinger, T. (2016, February 14). Economic uncertainty. Retrieved from Economics Help.org:https://www.economicshelp.org/blog/4941/economics/economic-uncertainty/
27. Public Health Service Act, Section 319. (n.d.). Public Health Emergencies. Retrieved from https://www.astho.org/: https://www.astho.org/Programs/Preparedness/Public-Health-Emergency-Law/Emergency-Authority-and-Imhttps://www.astho.org/Programs/Preparedness/Pmunity-Toolkit/Public-Health-Service-Act,-Section-319-Fact-Sheet/?terms=Public+Health+Service+Act%2c+Section+319
28. Richards, E. P., & Rathbun, K. C. (1999, July). The Role of the Police Power in 21st Century Public Health. Retrieved from journal laws:https://journals.1ww.com/stdjournal/fulltext/1999/07000/the_role_of_the_police_power_in_21st_century_.8.aspx
29. Rozenbaum, M. (2020, Jul. 6). the-increase-in-zoonotic-diseases-the-who-the-why-and-the-when. Retrieved from www.C.uk: https://www.C.uk/news/research-medical-benefits/the-increase-in-zoonotic-diseases-the-who-the-why-and-the-when/
30. Sartin, J. S. (1993, Apr. 16). 8513069. Retrieved from pubmed.ncbi.nlm.nih.gov:https://pubmed.ncbi.nlm.nih.gov/8513069/
31. Secretary Raimondo. (n.d.). Video Message from Secretary Raimondo. Retrieved from .commerce.gov:https://www.commerce.gov/about/leadership/gina-m-raimondo
32. sense, c. (n.d.). common-sense. Retrieved from https://dictionary.cambridge.org/: https://dictionary.cambridge.org/de/worterbuch/englisch/common-sense
33. SpendEdge. (2021, Jul. 27). organization-and-change-management-consulting-market-to-reach-usd-2-billion-by-2024-. Retrieved from SpendEdge: https://www.prnewswire.com/news-releases/organization-and-change-management-consulting-market-to-reach-usd-2-billion-by-2024--spendedge-301341217.html
34. Strategic goals. (n.d.). strategic-plan. Retrieved from commerce.go:https://www.commerce.gov/about/strategic-plan
35. Team Mighty. (2015, Dec. 21). 11-quotes-that-show-the-great-leadership-of-general-george-patton-2015-11. Retrieved from businessinsider.com: https://www.businessinsider.com/11-quotes-that-show-the-great-leadership-of-general-george-patton-2015-11?r=US&IR=T
36. Tuckman's stages of group development. (n.d.). Tuckman%27s_stages_of_group_development. Retrieved from wikipedia.org: https://en.wikipedia.org/wiki/Tuckman%27s_stages_of_group_development
37. Who Pandemic phase Descriptions and Main Action by Phase. (n.d.). documents. Retrieved from www.who.int:https://www.who.int/influenza/resources/documents/pandemic_phase_descriptions_and_actions.pdf
38. Wilkinson, D. (n.d.). /how-does-sms-work. Retrieved from simpletexting.com: https://simpletexting.com/how-does-sms-work/

What is claimed is:

1. A device for managing a spread of infectious diseases among users, wherein the device is associated with a user, wherein the device comprises:
a communication device configured for receiving one or more device data from one or more devices associated with one or more users, wherein the one or more device data comprises one or more location data associated with one or more locations of the one or more devices corresponding to one or more time periods;
a location sensor configured for generating location data based on a location of the device corresponding to a time period;
a pathogen tracer microcontroller device communicatively coupled with the communication device, wherein the pathogen tracer microcontroller device is configured for:
analyzing the location data and the one or more location data;
determining a spatiotemporal proximity of the device to the one or more devices based on the analyzing;
comparing the spatiotemporal proximity with a predetermined spatiotemporal proximity based on the determining of the spatiotemporal proximity;
determining a risk of the spread of a pathogen causing an infectious disease between the user and the one or more users based on the comparing; and
generating an alert based on the determining of the risk;
an output device communicatively coupled with the pathogen tracer microcontroller device, wherein the output device is configured for producing one or more alert signals based on the alert; and
a storage device communicatively coupled with the pathogen tracer microcontroller device, wherein the storage device is configured for:
storing the alert; and
storing a status of the user corresponding to the infectious disease.

2. The device of claim 1, wherein the pathogen tracer microcontroller device is further configured for identifying one or more first users from the one or more users based on the comparing, wherein the infectious disease is likely to spread between the user and the one or more first users, wherein the generating of the alert is further based on the identifying.

3. The device of claim 2, wherein the one or more device data comprises one or more statuses of the one or more users, wherein the pathogen tracer microcontroller device is further configured for identifying one or more first statuses of the one or more first users from the one or more statuses based on the identifying of the one or more first users, wherein the output device is further configured for displaying the one or more first statuses of the one or more first users.

4. The device of claim 3, wherein the storage device is further configured for retrieving the status of the user based on the determining of the spatiotemporal proximity, wherein the pathogen tracer microcontroller device is further configured for analyzing the status and the one or more statuses, wherein the determining of the risk is further based on the analyzing of the status and the one or more statuses.

5. The device of claim 4, wherein the pathogen tracer microcontroller device is further configured for:
updating at least one of the status and the one or more statuses based on the determining of the risk; and
generating at least one of an updated status of the user corresponding to the infectious disease and one or more updated statuses of the one or more users corresponding to the infectious disease based on the updating, wherein the storage device is further configured for storing at least one of the updated status and the one or more updated statuses.

6. The device of claim 5, wherein the communication device is further configured for transmitting at least one of the updated status and the one or more updated statuses to the one or more devices.

7. The device of claim 1, wherein the storage device is further configured for retrieving the status of the user based on the determining of the spatiotemporal proximity, wherein the communication device is further configured for transmitting the status to the one or more devices.

8. The device of claim 1, wherein the communication device is further configured for transmitting the alert to the one or more devices, wherein the one or more devices comprises one or more output devices, wherein the one or more output devices produces the one or more alert signals based on the alert.

9. The device of claim 1, wherein the one or more device data comprises one or more device identifiers of the one or more devices, one or more spatiotemporal identifiers of the one or more devices, and one or more statuses of the one or more users associated with the one or more devices, wherein the pathogen tracer microcontroller device is further configured for:
analyzing the one or more device identifiers, the one or more spatiotemporal identifiers, and the one or more statuses;
identifying one or more contaminated users from the one or more users based on the analyzing of the one or more device identifiers, the one or more spatiotemporal identifiers, and the one or more statuses; and
generating a first alert for the one or more contaminated users based on the identifying of the one or more suspected users, wherein the output device is further configured for producing one or more first alert signals based on the first alert.

10. The device of claim 1, wherein the pathogen tracer microcontroller device is further configured for generating one or more recommendations to prevent the risk of the spread of the pathogen causing the infectious disease based on the determining of the risk, wherein the output device is configured for displaying the one or more recommendations to the user.

11. The device of claim 1, wherein the device comprises a wearable device, wherein the wearable device is configured to be worn on at least a part of a body of the user.

12. A device for managing a spread of infectious diseases among users, wherein the device is associated with a user, wherein the device comprises:
a communication device configured for receiving one or more device data from one or more devices associated with one or more users, wherein the one or more device data comprises one or more location data associated with one or more locations of the one or more devices corresponding to one or more time periods;
a location sensor configured for generating location data based on a location of the device corresponding to a time period;
a pathogen tracer microcontroller device communicatively coupled with the communication device, wherein the pathogen tracer microcontroller device is configured for:
analyzing the location data and the one or more location data;
determining a spatiotemporal proximity of the device to the one or more devices based on the analyzing;
comparing the spatiotemporal proximity with a predetermined spatiotemporal proximity based on the determining of the spatiotemporal proximity;
determining a risk of the spread of a pathogen causing an infectious disease between the user and the one or more users based on the comparing;
identifying one or more first users from the one or more users based on the comparing, wherein the infectious disease is likely to spread between the user and the one or more first users; and
generating an alert based on the determining of the risk and the identifying;
an output device communicatively coupled with the pathogen tracer microcontroller device, wherein the output device is configured for producing one or more alert signals based on the alert; and
a storage device communicatively coupled with the pathogen tracer microcontroller device, wherein the storage device is configured for:
storing the alert; and
storing a status of the user corresponding to the infectious disease.

13. The device of claim 12, wherein the one or more device data comprises one or more statuses of the one or more users, wherein the pathogen tracer microcontroller device is further configured for identifying one or more first statuses of the one or more first users from the one or more statuses based on the identifying of the one or more first users, wherein the output device is further configured for displaying the one or more first statuses of the one or more first users.

14. The device of claim 13, wherein the storage device is further configured for retrieving the status of the user based on the determining of the spatiotemporal proximity, wherein the pathogen tracer microcontroller device is further configured for analyzing the status and the one or more statuses, wherein the determining of the risk is further based on the analyzing of the status and the one or more statuses.

15. The device of claim 14, wherein the pathogen tracer microcontroller device is further configured for:
updating at least one of the status and the one or more statuses based on the determining of the risk; and
generating at least one of an updated status of the user corresponding to the infectious disease and one or more updated statuses of the one or more users corresponding to the infectious disease based on the updating, wherein the storage device is further configured for storing at least one of the updated status and the one or more updated statuses.

16. The device of claim 15, wherein the communication device is further configured for transmitting at least one of the updated status and the one or more updated statuses to the one or more devices.

17. The device of claim 11, wherein the storage device is further configured for retrieving the status of the user based on the determining of the spatiotemporal proximity, wherein the communication device is further configured for transmitting the status to the one or more devices.

18. The device of claim 11, wherein the communication device is further configured for transmitting the alert to the one or more devices, wherein the one or more devices comprises one or more output devices, wherein the one or more output devices produces the one or more alert signals based on the alert.

19. The device of claim 11, wherein the one or more device data comprises one or more device identifiers of the one or more devices, one or more spatiotemporal identifiers of the one or more devices, and one or more statuses of the one or more users associated with the one or more devices, wherein the pathogen tracer microcontroller device is further configured for:
analyzing the one or more device identifiers, the one or more spatiotemporal identifiers, and the one or more statuses;
identifying one or more contaminated users from the one or more users based on the analyzing of the one or more device identifiers, the one or more spatiotemporal identifiers, and the one or more statuses; and
generating a first alert for the one or more contaminated users based on the identifying of the one or more suspected users, wherein the output device is further configured for producing one or more first alert signals based on the first alert.

20. The device of claim 11, wherein the pathogen tracer microcontroller device is further configured for generating one or more recommendations to prevent the risk of the spread of the pathogen causing the infectious disease based on the determining of the risk, wherein the output device is configured for displaying the one or more recommendations to the user.
